(19) United States
(12) Patent Application Publication
(10) Pub. No.: US 2021/0346313 A1
(43) Pub. Date: Nov. 11, 2021

(54) OPHTHALMIC COMPOSITIONS FOR TREATMENT OF OCULAR SURFACE DAMAGE AND SYMPTOMS OF DRYNESS

(71) Applicant: Novaliq GmbH, Heidelberg (DE)

(72) Inventors: Markus BEIER, Weinheim (DE); Daniela WILLEN, Oberzent (DE); Sonja KRÖSSER, Heidelberg (DE); Thomas SCHLÜTER, Heidelberg (DE)

(21) Appl. No.: 17/278,585

(22) PCT Filed: Sep. 20, 2019
(86) PCT No.: PCT/EP2019/075406

§ 371 Date: Mar. 22, 2021

(30) Foreign Application Priority Data
Sep. 22, 2018 (EP) 18196126.9
Oct. 3, 2018 (EP) 18198440.2
Oct. 23, 2018 (EP) 18202041.2

(51) Int. Cl. A61K 31/02; A61K 9/00; A61P 27/04

(52) U.S. Cl. CPC A61K 31/02; A61P 27/04; A61K 9/0048

(57) ABSTRACT

The present disclosure provides methods of treatment using ophthalmic compositions, comprising 1-perfluorohexyloctane, which are useful in the treatment of ocular surface damage of the cornea and/or symptoms of dryness.

### FIELD

The present disclosure is in the field of ophthalmic compositions comprising 1-perfluorohexyloctane, which are useful in the treatment of ocular surface damage of the cornea and/or symptoms of dryness.

### BACKGROUND

Keratoconjunctivitis sicca, also known as dry eye disease (DED), or dysfunctional tear syndrome, is a multifunctional disorder of the tear film, and ocular surface which results in discomfort, visual disturbance, and often even in ocular surface damage. Its prevalence differs widely by regions and is estimated to range from about 7.4% in the USA to about 33% in Japan (J. L. Gayton, Clinical Ophthalmology 2009:3, 405-412). According to another estimate, approximately 3.2 million women and 1.05 million men suffer from keratoconjunctivitis sicca in the USA alone. If symptomatically mild cases are also considered, there could be as many as 20 million affected people in the USA. Two major categories of dry eye disease (DED) are distinguished today, which are aqueous-deficient DED and evaporative DED. These conditions are not necessarily mutually exclusive.

Evaporative DED, is somewhat heterogeneous and can develop as a result of diverse root causes. Causes associated with increased evaporative loss of the tear film include Meibomian gland disease or dysfunction, eyelid aperture disorders, blink disorders (as in Parkinson disease) or ocular surface disorders (as in allergic conjunctivitis). In particular, Meibomian gland diseases and dysfunctions are prevalently associated with evaporative dry eye disease. For example, Meibomian gland dysfunction (also abbreviated as MGD) can result in changes in the quantitative or qualitative secretion of the lipid components required for the tear film. The meibum can also have an altered composition, enriched in some components and/or deficient in other components, compared to normal meibum. This may result in altered physical properties, such as abnormal viscosity or abnormal solubility. This in turn can lead to a failure in forming a stable and continuous tear film, which is followed by evaporative loss and hyperosmolarity. Meibomian gland dysfunction can often be characterized by gland obstruction and clogging through hyperkeratinisation of the gland and increased viscosity of the meibum. Dysfunction can arise from a primary lid-margin related disease or a secondary disease arising from systemic disorders such as acne rosacea or seborrheic dermatitis.

The mainstay of non-pharmacological DED treatment is the use of artificial tears for tear substitution. Most of the available products are designed as lubricants. In addition, they may function as carriers for nutrients and electrolytes (importantly, potassium and bicarbonate), and some products attempt to correct physical parameters such as an increased osmolarity in certain forms of DED.

Preservatives which can be used in ophthalmic formulations are potentially damaging to the eye, in particular to the ocular surface, and should be avoided in the context of dry eye disease. This is particularly relevant for patients with moderate to severe dry eye disease symptoms who may require frequent use for symptom relief, as well as patients who require multiple preserved topical medicaments.

WO 2011/073134 discloses ophthalmic topical pharmaceutical compositions comprising immunosuppressant macrolides such as ciclosporin A and semifluorinated alkanes, for treatment of keratoconjunctivitis sicca. The semifluorinated alkanes in the disclosed compositions serve as suitable liquid vehicles for delivering the therapeutic pharmaceutical agent to the eye, and in particular have a high capacity for dissolving poorly soluble compounds such as ciclosporin. In this role, however, the semifluorinated alkane is merely taught as pharmaceutically inactive solvent for the active therapeutic agent.

U.S. Pat. No. 7,001,607 discloses a polyaphron gel tear substitute containing at least one water-soluble fluorinated surfactant, water, and a non-polar component, in which the nonpolar component can be fluorocarbon or a silicone oil. The gel compositions are specifically administered into the conjunctival sac to form a gel reservoir, and are only spread over the cornea of the eye as a liquid film over the cornea as a result of blinking action. For patients with dry eye symptoms caused by eyelid/blink disorders (e.g. as a result of Parkinson's disease), such compositions are therefore not useful.

US 2015-0224064A1 discloses semifluorinated alkane compositions for the treatment of dry eye disease, as well as symptoms and conditions associated therewith. The disclosed invention is directed primarily to compositions comprising a mixture of at least two different semifluorinated alkanes. These compositions may be administered to the eye or ophthalmic tissues, such as, in patients suffering from keratoconjunctivitis sicca and/or Meibomian gland dysfunction. The publication does not disclose or suggest any method of providing an enrichment of semifluorinated alkane in the ophthalmic tissues or delayed ophthalmic release of semifluorinated alkane.

It is therefore an object of the present disclosure, to provide a composition for use in an improved, and more efficient method for the treatment of keratoconjunctivitis sicca, and/or keratoconjunctivitis sicca associated with Meibomian gland dysfunction and/or Meibomian gland dysfunction.

### BRIEF SUMMARY

In a first aspect, the present disclosure provides a method of treating (reducing) the ocular surface damage of one or more regions of the cornea, wherein the one or more regions of the cornea are selected from the group consisting of the total corneal region, the central corneal region, the nasal corneal region, the temporal corneal region, the inferior corneal region and combinations thereof.

In second aspect, the present disclosure provides a method of treating (reducing) one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof.

In a third aspect, the present disclosure provides a method of treating (reducing) the ocular surface damage of one or more regions of the cornea and of treating (reducing) one or more symptoms of dryness, wherein the one or more regions of the cornea are selected from the group consisting of the total corneal region, the central corneal region, the nasal corneal region, the temporal corneal region, the inferior corneal region and combinations thereof, and wherein the one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof.

In a fourth aspect, the present disclosure provides a method of treating ocular surface nerve sensation or one or more symptoms related thereto.

In a fifth aspect, the present disclosure provides a composition for use in a method according to the first aspect of the disclosure.

In a sixth aspect, the present disclosure provides a composition for use in a method according to the second aspect of the disclosure.

In a seventh aspect, the present disclosure provides a composition for use in a method according to the third aspect of the disclosure.

In an eighth aspect, the present disclosure provides a composition for use in a method according to the fourth aspect of the disclosure.

### DETAILED DESCRIPTION

In a first aspect embodiments of the present disclosure provide a method (Method 1) of treating (reducing) the ocular surface damage of one or more regions of the cornea, wherein the one or more regions of the cornea are selected from the group consisting of the total corneal region, the central corneal region, the nasal corneal region, the temporal corneal region, the inferior corneal region and combinations thereof, and wherein the method comprises the step of administering for up to 4 times per day a single drop of about 10-12 μl of a composition essentially consisting (or consisting of) of 1-perfluorohexyloctane, and optionally up to about 3 wt % of 2-perfluorohexyloctane, to the eye of a patient in need thereof. Further embodiments of the present disclosure provide as follows:
1.1 Method 1, wherein the composition essentially consists of 1-perfluorohexyloctane, and optionally up to about 1 wt % of 2-perfluorohexyloctane.
1.2 Method 1 or 1.1, wherein the composition essentially consists of 1-perfluorohexyloctane
1.3 Method 1 or any of 1.1 to 1.2, wherein the composition is administered as a single drop of 10-11 μl, preferably as a single drop of about 11 μl to the eye of a patient.
1.4 Method 1 to 1.3, wherein the composition is administered four times per day to the eye of a patient.
1.5 Method 1 or any of 1.1 to 1.4, wherein the method is effective in treating (reducing) the ocular surface damage of the total corneal region and/or the central corneal region and/or the nasal corneal region and/or the temporal corneal region and/or the inferior corneal region.
1.6 Method 1 or any of 1.1 to 1.5, wherein the method is effective within 2, 4 or 8 weeks after first administration of the composition to the eye of a patient.
1.7 Method 1 or any of 1.1 to 1.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
1.8 Method 1 or any of 1.1 to 1.7, wherein the ocular surface damage is not originating from cataract surgery.
1.9 Method 1 or any of 1.1 to 1.8, wherein the ocular surface damage is determined by grading one or more of the corneal regions selected from the group consisting of the total corneal region, the central corneal region, the nasal corneal region, the temporal corneal region and the inferior corneal region by fluorescein staining of the cornea.
1.10 Method 1.9, wherein the grading is performed according to the National Eye Institute scale.
1.11 Method 1 or any of 1.1 to 1.10, wherein the method is effective in treating (reducing) the ocular surface damage
   i. of the total and the central corneal region
   ii. of the total and the inferior corneal region
   iii. of the total and the nasal corneal region
   iv. of the total and the temporal corneal region
   v. of the central and the inferior corneal region
   vi. of the central and the nasal corneal region
   vii. of the central and the temporal region
   viii. of the inferior and the nasal region, or
   ix. of the inferior and the temporal region
1.12 Method 1 or any of 1.1 to 1.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2
1.13 Method 1 or any of 1.1 to 1.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
1.14 Method 1 or any of 1.1 to 1.13, wherein the method is effective in treating (reducing)
   i. the ocular surface damage of the total corneal region in a patient characterized by a tear film breakup time (TBUT) of lower than 3 s.
   ii. the ocular surface damage of the total corneal region in a patient characterized by a MGD score of equal or higher than 7
   iii. the ocular surface damage of the central corneal region in a patient characterized by a tear film breakup time (TBUT) of lower than 3 s.
   iv. the ocular surface damage of the central corneal region in a patient characterized by a MGD score of equal or higher than 7
1.15 Method 1 or any of 1.1 to 1.14, wherein the patient is a female
1.16 Method 1 or any of 1.1 to 1.14, wherein the patient is a male
1.17 Method 1 or any of 1.1 to 1.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
1.18 Method 1 or any of 1.1 to 1.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
1.19 Method 1 or any of 1.1 to 1.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
1.20 Method 1 or any of 1.1 to 1.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
1.21 Method 1 or any of 1.1 to 1.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
1.22 Method 1 or any of 1.1 to 1.21, wherein the patient is a contact lens wearer.
1.23 Method 1 or any of 1.1 to 1.22, wherein the patient was unresponsive or insufficiently response to previous treatment for keratoconjunctivitis sicca (dry eye disease).
1.24 Method 1.23, wherein said previous treatment comprise one or more of the following treatment methods: topical aqueous immunosuppressant administration (e.g., topical aqueous ciclosporin), topical corticosteroid administration, or topical aqueous artificial tears administration.
1.25 Method 1 or any of 1.1 to 1.24, wherein the method is effective in reducing the ocular surface damage
   i. of the total corneal region by at least 3 grades and/or
   ii. of the central corneal region by at least 1 grade as determined by grading the corneal regions by fluorescein staining of the cornea according to the National Eye Institute scale.
1.26 Method 1.25, wherein the method is effective within 2 weeks, preferably within 4 weeks, more preferably within 8 weeks of treatment.

In another aspect the present disclosure provides a composition essentially consisting of 1-perfluorohexyloctane, and optionally up to about 3 wt % of 2-perfluorohexyloctane, for use in Method 1 or any of their subsequent embodiments (i.e. Method 1.1 to 1.24).

In still another aspect, the present disclosure provides for the use of composition, as defined in Method 1 and their subsequent embodiments (Method 1.1 to 1.24) in the preparation or manufacture of a topically administered ophthalmic medicine or medicament.

As understood herein, the phrase ‘essentially consists of’ or ‘essentially consisting of’ and the phrase ‘consists of’ or ‘consisting of’ are considered to be interchangeable, and means that no further components are featured in the composition or dosage form, other than those listed, with the exception of, if present, negligible amount of material-inherent impurities which do not provide any technical contribution or function in regards to the disclosed composition or dosage form. The term ‘comprises’ or ‘comprising’, as used herein is in contrast, to be construed in an open sense, where features, for example composition components, other than those prefaced by the term may be present.

The terms ‘about’, ‘substantially’ ‘essentially’ and the like in connection with an attribute or value such as dose amount, or concentration as used herein includes the exact attribute or precise value, as well as any attribute, or value typically considered to fall within a normal range or accepted variability associated with the technical field and methods of measurement or determination of said attribute or value.

In a second aspect the present disclosure provides method (Method 2) of treating (reducing) one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof, wherein the method comprises the step of administering for up to 4 times per day a single drop of about 10-12 μl of a composition consisting of (or essentially consisting of) 1-perfluorohexyloctane, and optionally up to about 3 wt % of 2-perfluorohexyloctane, to the eye of a patient in need thereof. Further embodiments of the present disclosure provide as follows:
2.1 Method 2, wherein the composition essentially consists of 1-perfluorohexyloctane, and optionally up to about 1 wt % of 2-perfluorohexyloctane.
2.2 Method 2 or 2.1, wherein the composition essentially consists of 1-perfluorohexyloctane
2.3 Method 2 or any of 2.1 to 2.2, wherein the composition is administered as a single drop of 10-11 μl, preferably as a single drop of about 11 μl to the eye of a patient.
2.4 Method 2 or any of 2.1 to 2.3, wherein the composition is administered four times per day to the eye of a patient.
2.5 Method 2 or any of 2.1 to 2.4, wherein, the method is effective in treating (reducing) one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof.
2.6 Method 2 or any of 2.1 to 2.5, wherein, the method is effective within 2, 4 or 8 weeks after first administration of the composition to the eye of a patient.
2.7 Method 2 or any of 2.1 to 2.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
2.8 Method 2 or any of 2.1 to 2.7, wherein the ocular surface damage is not originating from cataract surgery.
2.9 Method 2 or any of 2.1 to 2.8, wherein the reduction of severity of dryness and/or blurred vision and/or sensitivity of light is determined by the Eye Dryness Score on a visual analog scale (VAS) from 0% to 100% indicating the level of discomfort of the patient
2.10 Method 2 or any of 2.1 to 2.9, wherein the reduction of frequency of dryness and/or the awareness of dryness is determined by the Eye Dryness Score on a visual analog scale (VAS) from 0% to 100% indicating the percentage of time said dryness symptoms are experienced by the patient.
2.11 Method 2 or any of 2.1 to 2.10, wherein the total ocular surface disease index (OSDI) score is determined on a scale of 1 to 100 with higher scores representing greater disability of the patient.
2.12 Method 2 or any of 2.1 to 2.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2;
   or wherein the patient to be treated is characterized by one or more, or all of the following criteria:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2,
   iv. a MGD score between 4.0 and 11.2, and
   v. a Schirmer I Test of equal or greater than 5 mm; or greater than 5 mm; or greater than 6, 7, 8, or 9 mm.
2.13 Method 2 or any of 2.1 to 2.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
   v. Schirmer I Test of equal or greater than 10 mm; or greater than 10 mm; or equal or greater than 15 mm; or equal or greater than 20 mm.
2.14 Method 2 or any of 2.1 to 2.13, wherein the method is effective in treating (reducing)
   i. the severity of dryness in a patient characterized by a MGD score of equal or higher than 7
   ii. the frequency of dryness in a patient characterized by a MGD score of higher than 7
2.15 Method 2 or any of 2.1 to 2.14, wherein the patient is a female
2.16 Method 2 or any of 2.1 to 2.14, wherein the patient is a male
2.17 Method 2 or any of 2.1 to 2.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
2.18 Method 2 or any of 2.1 to 2.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
2.19 Method 2 or any of 2.1 to 2.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
2.20 Method 2 or any of 2.1 to 2.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.

2.21 Method 2 or any of 2.1 to 2.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
2.22 Method 2 or any of 2.1 to 2.21, wherein the patient is a contact lens wearer.
2.23 Method 2 or any of 2.1 to 2.22, wherein the patient was unresponsive or insufficiently response to previous treatment for keratoconjunctivitis sicca (dry eye disease).
2.24 Method 2.23, wherein said previous treatment comprise one or more of the following treatment methods: topical aqueous immunosuppressant administration (e.g., topical aqueous ciclosporin), topical corticosteroid administration, or topical aqueous artificial tears administration.

In one embodiment of Method 2 or any of its subsequent embodiments (i.e. Method 2.1 to 2.24), provided is a method for the treatment of (the symptom) severity of dryness in a patient suffering from dry eye disease associated with Meibomian Gland Dysfunction, where the method comprises the step of administering for up to 4 times per day a single drop of about 10-12 μl of a composition consisting of (or essentially consisting of) 1-perfluorohexyloctane, and optionally up to about 3 wt % of 2-perfluorohexyloctane, to the eye of a patient in need thereof, and wherein the patient is characterized by a Schirmer I Test of equal or greater than 10 mm.

In another aspect the present disclosure provides a composition essentially consisting of 1-perfluorohexyloctane, and optionally up to about 3 wt % of 2-perfluorohexyloctane, for use in Method 2 or any of their subsequent embodiments (i.e. Method 2.1 to 2.24).

In still another aspect, the present disclosure provides for the use of composition as defined in Method 2 and their subsequent embodiments (Method 2.1 to 2.24) in the preparation or manufacture of a topically administered ophthalmic medicine or medicament.

In a third aspect the present disclosure provides a method (Method 3) of treating (reducing) the ocular surface damage of one or more regions of the cornea and of treating (reducing) one or more symptoms of dryness, wherein the one or more regions of the cornea are selected from the group consisting of the total corneal region, the central corneal region, the nasal corneal region, the temporal corneal region, the inferior corneal region and combinations thereof, and wherein the one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof, and wherein the method comprises the step of administering for up to 4 times per day a single drop of about 10-12 μl of a composition consisting of (or essentially consisting of) 1-perfluorohexyloctane, and optionally up to about 3 wt % of 2-perfluorohexyloctane, to the eye of a patient in need thereof. Further embodiments of the present disclosure provide as follows:
3.1 Method 3, wherein the composition essentially consists of 1-perfluorohexyloctane, and optionally up to about 1 wt % of 2-perfluorohexyloctane.
3.2 Method 3 or 3.1, wherein the composition essentially consists of 1-perfluorohexyloctane
3.3 Method 3 or any of 3.1 to 3.2, wherein the composition is administered as a single drop of 10-11 μl, preferably as a single drop of about 11 μl to the eye of a patient.
3.4 Method 3 or any of 3.1 to 3.3, wherein the composition is administered four times per day to the eye of a patient.
3.5 Method 3 or any of 3.1 to 3.4, wherein the method is effective intreating (reducing) the ocular surface damage of one or more regions of the cornea and effective in treating (reducing) one or more symptoms of dryness, wherein the one or more regions of the cornea are selected from the group consisting of the total corneal region, the central corneal region, the nasal corneal region, the temporal corneal region, the inferior corneal region and combinations thereof, and wherein the one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof.
3.6 Method 3 or any of 3.1 to 3.5, wherein the method is effective within 2, 4 or 8 weeks after first administration of the composition to the eye of a patient.
3.7 Method 3 or any of 3.1 to 3.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
3.8 Method 3 or any of 3.1 to 3.7, wherein the ocular surface damage is not originating from cataract surgery.
3.9 Method 3 or any of 3.1 to 3.8, wherein the reduction of severity of dryness and/or blurred vision and/or sensitivity of light is determined by the Eye Dryness Score on a visual analog scale (VAS) from 0% to 100% indicating the level of discomfort of the patient
3.10 Method 3 or any of 3.1 to 3.9, wherein the reduction of frequency of dryness and/or the awareness of dryness is determined by the Eye Dryness Score on a visual analog scale (VAS) from 0% to 100% indicating the percentage of time said dryness symptoms are experienced.
3.11 Method 3 or any of 3.1 to 3.10, wherein the total ocular surface disease index (OSDI) score is determined on a scale of 1 to 100 with higher scores representing greater disability of the patient.
3.12 Method 3 or any of 3.1 to 3.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2;
   or wherein the patient to be treated is characterized by one or more, or all of the following criteria:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2,
   iv. a MGD score between 4.0 and 11.2, and
   v. a Schirmer I Test of equal, or greater than 5 mm; or greater than 5 mm; or greater than 6, 7, 8, or 9 mm.
3.13 Method 3 or any of 3.1 to 3.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
   v. Schirmer I Test of equal or greater than 10 mm; greater than 10 mm; equal or greater than 15 mm; or equal or greater than 20 mm.
3.14 Method 3 or any of 3.1 to 3.13, wherein the method is effective in treating (reducing) simultaneously the ocular damage of one or more corneal regions and the symptoms of dryness, preferably within 2, 4 or 8 weeks after first administration of the composition to the eye of a patient in need thereof.
3.15 Method 3 or any of 3.1 to 3.14, wherein the method is effective in treating (reducing) the ocular surface damage of the total corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.16 Method 3 or any of 3.1 to 3.14, wherein the method is effective in treating (reducing) the ocular surface damage of the central corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.17 Method 3 or any of 3.1 to 3.14, wherein the method is effective in treating (reducing) the ocular surface damage of the inferior corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.18 Method 3 or any of 3.1 to 3.14, wherein the method is effective in treating (reducing) the ocular surface damage of the nasal corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.19 Method 3 or any of 3.1 to 3.14, wherein the method is effective in treating (reducing) the ocular surface damage of the temporal corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.20 Method 3 or any of 3.1 to 3.14, wherein the method is effective in treating (reducing) the ocular surface damage of the total and the central corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.21 Method 3 or any of 3.1 to 3.14, wherein the method is effective in treating (reducing) the ocular surface damage of the central and the inferior corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
3.22 Method 3 or any of 3.1 to 3.21, wherein the patient is a female
3.23 Method 3 or any of 3.1 to 3.21, wherein the patient is a male
3.24 Method 3 or any of 3.1 to 3.23, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
3.25 Method 3 or any 3.1 to 3.24, wherein the ocular surface damage is determined by grading one or more of the corneal regions selected from the group consisting of the total corneal region, the central corneal region, the nasal corneal region, the temporal corneal region and the inferior corneal region by fluorescein staining of the cornea
3.26 Method 3 or any of 3.1 to 3.25, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
3.27 Method 3 or any of 3.1 to 3.26, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
3.28 Method 3 or any of 3.1 to 3.27, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
3.29 Method 3 or any of 3.1 to 3.28, wherein the patient is concomitantly under treatment with another topical ophthalmic medication, for example, an antibiotic, antifungal, corticosteroid, immunosuppressant, sympathomimetic, anesthetic, antihistamine, or any combination thereof.
3.30 Method 3 or any of 3.1 to 3.29, wherein the patient is a contact lens wearer.
3.31 Method 3 or any of 3.1 to 3.30, wherein the patient was unresponsive or insufficiently response to previous treatment for keratoconjunctivitis sicca (dry eye disease).
3.32 Method 3.31, wherein said previous treatment comprise one or more of the following treatment methods: topical aqueous immunosuppressant administration (e.g., topical aqueous ciclosporin), topical corticosteroid administration, or topical aqueous artificial tears administration.
3.33 Method 3 or 3.1 to 3.32, wherein the method is effective in reducing the ocular surface damage
   i. of the total corneal region by at least 3 grades and/or
   ii. of the central corneal region by at least 1 grade
   as determined by grading the corneal regions by fluorescein staining of the cornea according to the National Eye Institute scale.
3.34 Method 3.33, wherein the method is effective within 2 weeks, preferably within 4 weeks, more preferably within 8 weeks of treatment.

In one embodiment of Method 3, or any of its subsequent embodiments (i.e. Methods 3.1 to 3.34), is provided a method for treating (reducing) the ocular surface damage of one or more regions of the cornea and for treating (reducing) the symptom of the severity of dryness in a patient suffering from dry eye disease associated with Meibomian Gland Dysfunction, where the method comprises the step of administering for up to 4 times per day a single drop of about 10-12 μl of a composition consisting of (or essentially consisting of) 1-perfluorohexyloctane, and optionally up to about 3 wt % of 2-perfluorohexyloctane, to the eye of a patient in need thereof, and wherein the patient is characterized by a Schirmer I Test of equal or greater than 10 mm.

In another aspect, the present disclosure provides a composition essentially consisting of 1-perfluorohexyloctane, and optionally up to about 3 wt % of 2-perfluorohexyloctane, for use in Method 3 or any of their subsequent embodiments (i.e. Method 3.1 to 3.23).

In still another aspect, the present disclosure provides for the use of composition as defined in Method 3 and their subsequent embodiments (Method 3.1 to 3.23) in the preparation or manufacture of a topically administered ophthalmic medicine or medicament

In a fourth aspect the present disclosure provides a method (Method 4) of treating ocular surface nerve sensation or one or more symptoms related thereto, wherein the method comprises the step of administering for up to 4 times per day a single drop of about 10-12 μl of a composition consisting of (or essentially consisting of) 1-perfluorohexyloctane, optionally comprising up to about 1 wt % of 2-perfluorohexyloctane, to the eye of a patient in need thereof. Further embodiments of the present disclosure provide as follows:
4.1 Method 4, wherein the composition essentially consists of 1-perfluorohexyloctane, and optionally up to about 1 wt % of 2-perfluorohexyloctane.
4.2 Method 4 or 4.1, wherein the composition essentially consists of 1-perfluorohexyloctane
4.3 Method 4 or any of 4.1 to 4.2, wherein the composition is administered as a single drop of 10-11 μl, preferably as a single drop of about 11 μl to the eye of a patient.
4.4 Method 4 or any of 4.1 to 4.3, wherein the composition is administered four times per day to the eye of a patient.
4.5 Method 4 or any of 4.1 to 4.4, wherein the method is effective in treating ocular surface nerve sensation or one or more symptoms related thereto.
4.6 Method 4 or any of 4.1 to 4.5, wherein the method is effective in protecting the ocular surface nerves.
4.7 Method 4 or any of 4.1 to 4.6, wherein the composition is effective in treating (reducing) pathological signaling of the ocular surface nerves.
4.8 Method 4 or any of 4.1 to 4.7, wherein the method is effective within 2, 4 or 8 weeks after first administration of the composition to the eye of a patient.
4.9 Method 4 or any of 4.1 to 4.8, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
4.10 Method 4 or any of 4.1 to 4.9, wherein the ocular surface damage is not originating from cataract surgery.
4.11 Method 4 or any of 4.1 to 4.9, wherein the ocular surface damage is originating from cataract surgery.
4.12 Method 4 or any of 4.1 to 4.11, wherein the ocular surface nerves are selected from nerves at the surface of the cornea and/or the conjunctiva
4.13 Method 4 or any of 4.1 to 4.12, wherein the one or more symptoms associated with ocular surface nerve sensation are selected from eyes feeling gritty, eyes that are sensitive to light (photophobia), painful or sore eyes
4.14 Method 4 or any of 4.1 to 4.13, wherein the patient is characterized by
   i. one or more symptoms selected from eyes feeling gritty, eyes that are sensitive to light (photophobia) and/or painful or sore eyes, and
   ii. total corneal fluorescein staining (NEI scale) between 5 and 9
4.15 Method 4 or any of 4.1 to 4.14, wherein the patient is a female
4.16 Method 4 or any of 4.1 to 4.14, wherein the patient is a male
4.17 Method 4 or any of 4.1 to 4.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
4.18 Method 4 or any of 4.1 to 4.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
4.19 Method 4 or any of 4.1 to 4.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
4.20 Method 4 or any of 4.1 to 4.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
4.21 Method 4 or any of 4.1 to 4.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
4.22 Method 4 or any of 4.1 to 4.21, wherein the patient is a contact lens wearer.
4.23 Method 4 or any of 4.1 to 4.22, wherein the patient was unresponsive or insufficiently response to previous treatment for keratoconjunctivitis sicca (dry eye disease).
4.24 Method 4.23, wherein said previous treatment comprise one or more of the following treatment methods: topical aqueous immunosuppressant administration (e.g., topical aqueous ciclosporin), topical corticosteroid administration, or topical aqueous artificial tears administration.

In another aspect the present disclosure provides a composition essentially consisting of 1-perfluorohexyloctane, and optionally up to about 3 wt % of 2-perfluorohexyloctane, for use in Method 4 or any of their subsequent embodiments (i.e. Method 4.1 to 4.15).

In still another aspect, the present disclosure provides for the use of composition, as defined in Method 4 and their subsequent embodiments (Method 4.1 to 4.15) in the preparation or manufacture of a topically administered ophthalmic medicine or medicament.

In a fifth aspect, related to the first aspect, the present disclosure provides a composition for use (Composition for use 1) in the treatment of ocular surface damage of one or more regions of the cornea, wherein the one or more regions are selected from the group consisting of the total corneal region, the central corneal region, the nasal corneal region, the temporal corneal region, the inferior corneal region and combinations thereof, and wherein the composition essentially consists (or consists of) of 1-perfluorohexyloctane, and optionally up to about 3 wt % of 2-perfluorohexyloctane, and wherein the composition is administered for up to 4 times per day as a single drop of about 10-12 μl to the eye of a patient in need thereof. Further embodiments of the present disclosure provide as follows:
5.1 Composition for use 1, wherein the composition essentially consists of 1-perfluorohexyloctane, and optionally up to about 1 wt % of 2-perfluorohexyloctane.
5.2 Composition for use 1 or 5.1, wherein the composition essentially consists of 1-perfluorohexyloctane
5.3 Composition for use 1 or any of 5.1 to 5.2, wherein the composition is administered as a single drop of 10-11 μl, preferably as a single drop of about 11 μl to the eye of a patient.
5.4 Composition for use 1 or any of 5.1 to 5.3, wherein the composition is administered four times per day to the eye of a patient.
5.5 Composition for use 1 or any of 5.1 to 5.4, wherein the composition for use is effective in treating (reducing) the ocular surface damage of the total corneal region and/or the central corneal region and/or the nasal corneal region and/or the temporal corneal region and/or the inferior corneal region),
5.6 Composition for use 1 or any of 5.1 to 5.5, wherein the composition for use is effective within 2, 4 or 8 weeks after first administration of the composition to the eye of a patient.
5.7 Composition for use 1 or any of 5.1 to 5.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
5.8 Composition for use 1 or any of 5.1 to 5.7, wherein the ocular surface damage is not originating from cataract surgery.
5.9 Composition for use 1 or any of 5.1 to 5.8, wherein the ocular surface damage is determined by grading one or more of the corneal regions selected from the group consisting of the total corneal region, the central corneal region, the nasal corneal region, the temporal corneal region and the inferior corneal region by fluorescein staining of the cornea.
5.10 Composition for use 5.9, wherein the grading is performed according to the National Eye Institute scale.
5.11 Composition for use 1 or any of 5.1 to 5.10, wherein the composition for use is effective in treating (reducing) the ocular surface damage
   i. of the total and the central corneal region
   ii. of the total and the inferior corneal region
   iii. of the total and the nasal corneal region
   iv. of the total and the temporal corneal region
   v. of the central and the inferior corneal region
   vi. of the central and the nasal corneal region
   vii. of the central and the temporal region
   viii. of the inferior and the nasal region, or
ix. of the inferior and the temporal region
5.12 Composition for use 1 or any of 5.1 to 5.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2
5.13 Composition for use 1 or any of 5.1 to 5.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
5.14 Composition for use 1 or any of 5.1 to 5.13, wherein the composition for use is effective in treating (reducing)
   i. the ocular surface damage of the total corneal region in a patient characterized by a tear film breakup time (TBUT) of lower than 3 s.
   ii. the ocular surface damage of the total corneal region in a patient characterized by a MGD score of equal or higher than 7
   iii. the ocular surface damage of the central corneal region in a patient characterized by a tear film breakup time (TBUT) of lower than 3 s.
   iv. the ocular surface damage of the central corneal region in a patient characterized by a MGD score of equal or higher than 7
5.15 Composition for use 1 or any of 5.1 to 5.14, wherein the patient is a female
5.16 Composition for use 1 or any of 5.1 to 5.14, wherein the patient is a male
5.17 Composition for use 1 or any of 5.1 to 5.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
5.18 Composition for use 1 or any of 5.1 to 5.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
5.19 Composition for use 1 or any of 5.1 to 5.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
5.20 Composition for use 1 or any of 5.1 to 5.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
5.21 Composition for use 1 or any of 5.1 to 5.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
5.22 Composition for use 1 or any of 5.1 to 5.21, wherein the patient is a contact lens wearer.
5.23 Composition for use 1 or any of 5.1 to 5.22, wherein the patient was unresponsive or insufficiently response to previous treatment for keratoconjunctivitis sicca (dry eye disease).
5.24 Composition for use 5.23, wherein said previous treatment comprise one or more of the following treatment methods: topical aqueous immunosuppressant administration (e.g., topical aqueous ciclosporin), topical corticosteroid administration, or topical aqueous artificial tears administration.
5.25 Composition for use or any of 5.1 to 5.24, wherein the composition is effective in reducing the ocular surface damage
   i. of the total corneal region by at least 3 grades and/or
   ii. of the central corneal region by at least 1 grade as determined by grading the corneal regions by fluorescein staining of the cornea according to the National Eye Institute scale.
5.26 Composition for use 5.25, wherein the composition is effective within 2 weeks, preferably within 4 weeks, more preferably within 8 weeks of treatment.

In a sixth aspect, related to the second aspect, the present disclosure provides a composition for use (Composition for use 2) in the treatment of one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof, and wherein the composition essentially consists of (or consists of) 1-perfluorohexyloctane, and optionally up to about 3 wt % of 2-perfluorohexyloctane, and wherein the composition is administered for up to 4 times per day as a single drop of about 10-12 μl to the eye of a patient in need thereof. Further embodiments of the present disclosure provide as follows:
6.1 The Composition for use 2, wherein the composition essentially consists of 1-perfluorohexyloctane, and optionally up to about 1 wt % of 2-perfluorohexyloctane.
6.2 The Composition for use 2 or 6.1, wherein the composition essentially consists of 1-perfluorohexyloctane
6.3 The Composition for use 2 or any of 6.1 to 6.2, wherein the composition is administered as a single drop of 10-11 μl, preferably as a single drop of about 11 μl to the eye of a patient.
6.4 The Composition for use 2 or any of 6.1 to 6.3, wherein the composition is administered four times per day to the eye of a patient.
6.5 Composition for use 2 or any of 6.1 to 6.4, wherein, the composition for use is effective in the treatment (reduction) of one or more symptoms of dryness selected from the group consisting of severity of dryness, frequency of dryness, awareness of dryness, burning/stinging, itching, sticky feeling, blurred vision, foreign body sensation, total ocular surface disease index (OSDI) score and combinations thereof.
6.6 Composition for use 2 or any of 6.1 to 6.5, wherein, the composition for use is effective within 2, 4 or 8 weeks after first administration of the composition to the eye of a patient.
6.7 Composition for use 2 or any of 6.1 to 6.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
6.8 Composition for use 2 or any of 6.1 to 6.7, wherein the ocular surface damage is not originating from cataract surgery.
6.9 The Composition for use 2 or any of 6.1 to 6.8, wherein the reduction of severity of dryness and/or blurred vision and/or sensitivity of light is determined by the Eye Dryness Score on a visual analog scale (VAS) from 0% to 100% indicating the level of discomfort of the patient
6.10 The Composition for use 2 or any of 6.1 to 6.9, wherein the reduction of frequency of dryness and/or the awareness of dryness is determined by the Eye Dryness Score on a visual analog scale (VAS) from 0% to 100% indicating the percentage of time said dryness symptoms are experienced.
6.11 Composition for use 2 or any of 6.1 to 6.10, wherein the total ocular surface disease index (OSDI) score is determined on a scale of 1 to 100 with higher scores representing greater disability of the patient.
6.12 Composition for use 2 or any of 6.1 to 6.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2;
   or wherein the patient to be treated is characterized by one or more, or all of the following criteria:
   v. a tear film breakup time (TBUT) between 2.1 and 3.9 sec,
   vi. an ocular surface disease index (OSDI) of between 38 and 72,
   vii. a total corneal fluorescein staining (NEI scale) between 4.8 and 9.2,
   viii. a MGD score between 4.0 and 11.2, and
   ix. a Schirmer I Test of equal, or greater than 5 mm; or greater than 5 mm; or greater than 6, 7, 8, or 9 mm.
6.13 Composition for use 2 or any of 6.1 to 6.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
   v. Schirmer I Test of equal, or greater than 10 mm; greater than 10 mm; equal or greater than 15 mm; or equal or greater than 20 mm.
6.14 Composition for use 2 or any of 6.1 to 6.13, wherein the composition for use is effective in treating (reducing)
   i. the severity of dryness in a patient characterized by a MGD score of equal or higher than 7
   ii. the frequency of dryness in a patient characterized by a MGD score of higher than 7 6.15 Composition for use 2 or any of 6.1 to 6.14, wherein the patient is a female
6.16 Composition for use 2 or any of 6.1 to 6.14, wherein the patient is a male
6.17 Composition for use 2 or any of 6.1 to 6.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
6.18 Composition for use 2 or any of 6.1 to 6.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
6.19 Composition for use 2 or any of 6.1 to 6.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
6.20 Composition for use 2 or any of 6.1 to 6.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
6.21 Composition for use 2 or any of 6.1 to 6.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
6.22 Composition for use 2 or any of 6.1 to 6.21, wherein the patient is a contact lens wearer.
6.23 Composition for use 2 or any of 6.1 to 6.22, wherein the patient was unresponsive or insufficiently response to previous treatment for keratoconjunctivitis sicca (dry eye disease).
6.24 Composition for use 6.23, wherein said previous treatment comprise one or more of the following treatment methods: topical aqueous immunosuppressant administration (e.g., topical aqueous ciclosporin), topical corticosteroid administration, or topical aqueous artificial tears administration.

In one embodiment of the Composition for use 2 or any of its subsequent embodiments (i.e. Composition for use 6.1-6.24), is provided a composition for use in the treatment of the (symptom of) severity of dryness, wherein the composition essentially consists of (or consists of) 1-perfluorohexyloctane, and optionally up to about 3 wt % of 2-perfluorohexyloctane, and wherein the composition is administered for up to 4 times per day as a single drop of about 10-12 μl to the eye of a patient in need thereof, and wherein said patient is characterized by a Schirmer I Test of equal or greater than 10 mm.

In a seventh aspect, related to the third aspect, the present disclosure further provides a composition for use (Composition for use 3) in the treatment of the ocular surface damage of one or more regions of the cornea and for use in the treatment of one or more symptoms of dryness, wherein the one or more regions of the cornea are selected from the group consisting of the total corneal region, the central corneal region, the nasal corneal region, the temporal corneal region, the inferior corneal region and combinations thereof, and wherein the one or more symptoms of dryness are selected from the group consisting of severity of dryness, frequency of dryness, awareness of dryness, burning/stinging, itching, sticky feeling, blurred vision, foreign body sensation, total ocular surface disease index (OSDI) score and combinations thereof, and wherein the composition essentially consists of (or essentially consists of) 1-perfluorohexyloctane, and optionally up to about 3 wt % of 2-perfluorohexyloctane, and wherein the composition is administered for up to 4 times per day as a single drop of about 10-12 μl to the eye of a patient in need thereof. Further embodiments of the present disclosure provide as follows:
7.1 Composition for use 3, wherein the composition essentially consists of 1-perfluorohexyloctane, and optionally up to about 1 wt % of 2-perfluorohexyloctane.
7.2 Composition for use 3 or 7.1, wherein the composition essentially consists of 1-perfluorohexyloctane
7.3 Composition for use 3 or any of 7.1 to 7.2, wherein the composition is administered as a single drop of 10-11 μl, preferably as a single drop of about 11 μl to the eye of a patient.
7.4 Composition for use 3 or any of 7.1 to 7.3, wherein the composition is administered four times per day to the eye of a patient
7.5 Composition for use 3 or any of 7.1 to 7.4, wherein the composition for use is effective in the treatment of the ocular surface damage of one or more regions of the cornea and wherein the composition for use is effective in the treatment of one or more symptoms of dryness, wherein the one or more regions of the cornea are selected from the group consisting of the total corneal region, the central corneal region, the nasal corneal region, the temporal corneal region, the inferior corneal region and combinations thereof, and wherein the one or more symptoms of dryness are selected from the group consisting of severity of dryness, frequency of dryness, awareness of dryness, burning/stinging, itching, sticky feeling, blurred vision, foreign body sensation, total ocular surface disease index (OSDI) score and combinations thereof.
7.6 Composition for use 3 or any of 7.1 to 7.5, wherein the composition for use is effective within 2, 4 or 8 weeks after first administration of the composition to the eye of a patient.
7.7 Composition for use 3 or any of 7.1 to 7.6, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
7.8 Composition for use 3 or any of 7.1 to 7.7, wherein the ocular surface damage is not originating from cataract surgery.
7.9 The Composition for use 3 or any of 7.1 to 7.8, wherein the reduction of severity of dryness and/or blurred vision and/or sensitivity of light is determined by the Eye Dryness Score on a visual analog scale (VAS) from 0% to 100% indicating the level of discomfort of the patient.
7.10 The Composition for use 3 or any of 7.1 to 7.8, wherein the reduction of frequency of dryness and/or the awareness of dryness is determined by the Eye Dryness Score on a visual analog scale (VAS) from 0% to 100% indicating the percentage of time said dryness symptoms are experienced by the patient.
7.11 Composition for use 3 or any of 7.1 to 7.10, wherein the total ocular surface disease index (OSDI) score is determined on a scale of 1 to 100 with higher scores representing greater disability of the patient.
7.12 Composition for use 3 or any of 7.1 to 7.11, wherein the patient to be treated is characterized by:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2, and
   iv. a MGD score between 4.0 and 11.2;
   or wherein the patient to be treated is characterized by one or more, or all of the following criteria:
   i. a tear film breakup time (TBUT) between 2.1 and 3.9 sec,
   ii. an ocular surface disease index (OSDI) of between 38 and 72,
   iii. a total corneal fluorescein staining (NEI scale) between 4.8 and 9.2,
   iv. a MGD score between 4.0 and 11.2, and
   v. a Schirmer I Test of equal, or greater than 5 mm; or greater than 5 mm; or greater than 6, 7, 8, or 9 mm.
7.13 Composition for use 3 or any of 7.1 to 7.12, wherein the patient to be treated is characterized by one or more criteria selected from:
   i. a tear film breakup time (TBUT) of lower than 3 sec,
   ii. an ocular surface disease index (OSDI) of higher than 57,
   iii. a total corneal fluorescein staining (NEI scale) between 5 and 9
   iv. a MGD score of equal or higher than 7
   v. Schirmer I Test of equal or greater than 10 mm; equal or greater than 15 mm; equal or greater than 20 mm.
7.14 The Composition for use 3 or any of 7.1 to 7.13, wherein the composition for use is effective in treating (reducing) simultaneously the ocular surface damage of one or more regions of the cornea and is effective in treating (reducing) one or more symptoms of dryness, preferably within 2, 4 or 8 weeks after first administration of the composition to the eye of a patient in need thereof.
7.15 Composition for use 3 or any of 7.1 to 7.14, wherein the composition for use is effective in treating (reducing) the ocular surface damage of the total corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.16 Composition for use 3 or any of 7.1 to 7.14, wherein the composition for use is effective in treating (reducing) the ocular surface damage of the central corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.17 Composition for use 3 or any of 7.1 to 7.14, wherein the composition for use is effective in treating (reducing) the ocular surface damage of the inferior corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.18 Composition for use 3 or any of 7.1 to 7.14, wherein the composition for use is effective in treating (reducing) the ocular surface damage of the nasal corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.19 Composition for use 3 or any of 7.1 to 7.14, wherein the composition for use is effective in treating (reducing) the ocular surface damage of the temporal corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.20 Composition for use 3 or any of 7.1 to 7.14, wherein the composition for use is effective in treating (reducing) the ocular surface damage of the total and the central corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.21 Composition for use 3 or any of 7.1 to 7.14, wherein the composition for use is effective in treating (reducing) the ocular surface damage of the central and the inferior corneal region, and is effective in treating (reducing) one or more symptoms of dryness selected from the group consisting of severity of dryness, frequency of dryness, awareness of dryness, burning/stinging, itching, sticky feeling, blurred vision, foreign body sensation total ocular surface disease index (OSDI) score and combinations thereof.
7.22 Composition for use 3 or any of 7.1 to 7.21, wherein the patient is a female
7.23 Composition for use 3 or any of 7.1 to 7.21, wherein the patient is a male
7.24 Composition for use 3 or any of 7.1 to 7.23, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
7.25 Composition for use 3 or any of 7.1 to 7.24, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
7.26 Composition for use 3 or any of 7.1 to 7.25, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
7.27 Composition for use 3 or any of 7.1 to 7.26, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
7.28 Composition for use 3 or any of 7.1 to 7.27, wherein the patient is concomitantly under treatment with another topical ophthalmic medication, for example, an antibiotic, antifungal, corticosteroid, immunosuppressant, sympathomimetic, anesthetic, antihistamine, or any combination thereof.
7.29 Composition for use 3 or any of 7.1 to 7.28, wherein the patient is a contact lens wearer.
7.30 Composition for use 3 or any of 7.1 to 7.29, wherein the patient was unresponsive or insufficiently response to previous treatment for keratoconjunctivitis sicca (dry eye disease).
7.31 Composition for use 7.30, wherein said previous treatment comprise one or more of the following treatment methods: topical aqueous immunosuppressant administration (e.g., topical aqueous ciclosporin), topical corticosteroid administration, or topical aqueous artificial tears administration.
7.32 Composition for use 3 or any of 7.1 to 7.31, wherein the method is effective in reducing the ocular surface damage
   i. of the total corneal region by at least 3 grades and/or
   ii. of the central corneal region by at least 1 grade as determined by grading the corneal regions by fluorescein staining of the cornea according to the National Eye Institute scale.
7.33 Composition for use 7.32, wherein the composition is effective within 2 weeks, preferably within 4 weeks, more preferably within 8 weeks of treatment.

In one embodiment of the Composition for use 3 or any of its subsequent embodiments (i.e. Composition for use 7.1 to 7.33), is provided a composition for use in the treatment (reduction) of the ocular surface damage of one or more regions of the cornea and for the treatment (reduction) of the (symptom of) severity of dryness, wherein the composition essentially consists of (or consists of) 1-perfluorohexyloctane, and optionally up to about 3 wt % of 2-perfluorohexyloctane, and wherein the composition is administered for up to 4 times per day as a single drop of about 10-12 μl to the eye of a patient in need thereof, and wherein said patient is characterized by a Schirmer I Test of equal or greater than 10 mm.

In an eighth aspect, related to the fourth aspect, the present disclosure further provides a composition for use (Composition for use 4) in the treatment (reduction) of ocular surface nerve sensation or one or more symptoms related thereto, wherein the method comprises the step of administering for up to 4 times per day a single drop of about 10-12 μl of a composition consisting of (or essentially consisting of) 1-perfluorohexyloctane, optionally comprising up to about 1 wt % of 2-perfluorohexyloctane, to the eye of a patient in need thereof. Further embodiments of the present disclosure provide as follows:
8.1 Composition for use 4, wherein the composition essentially consists of 1-perfluorohexyloctane, and optionally up to about 1 wt % of 2-perfluorohexyloctane.
8.2 Composition for use 4 or 8.1, wherein the composition essentially consists of 1-perfluorohexyloctane
8.3 Composition for use 4 or any of 8.1 to 8.2, wherein the composition is administered as a single drop of 10-11 μl, preferably as a single drop of about 11 μl to the eye of a patient.
8.4 Composition for use 4 or any of 8.1 to 8.3, wherein the composition is administered four times per day to the eye of a patient.
8.5 Composition for use 4 or any of 8.1 to 8.4, wherein the composition for use is effective in treating ocular surface nerve sensation or one or more symptoms related thereto.
8.6 Composition for use 4 or any of 8.1 to 8.5, wherein the composition for use is effective in protecting the ocular surface nerves
8.7 Composition for use 4 or any of 8.1 to 8.6, wherein the composition for use is effective in treating (reducing) pathological signaling of the ocular surface nerves.
8.8 Composition for use 4 or any of 8.1 to 8.7, wherein the composition for use is effective within 2, 4 or 8 weeks after first administration of the composition to the eye of a patient.
8.9 Composition for use 4 or any of 8.1 to 8.8, wherein the patient suffers from keratoconjunctivitis sicca (dry eye disease) and/or keratoconjunctivitis sicca (dry eye disease) associated with Meibomian gland dysfunction and/or evaporative keratoconjunctivitis sicca (dry eye disease) associated with Meibomian gland dysfunction and/or Meibomian gland dysfunction.
8.10 Composition for use 4 or any of 8.1 to 8.9, wherein the ocular surface damage is not originating from cataract surgery.
8.11 Composition for use 4 or any of 8.1 to 8.9, wherein the ocular surface damage is originating from cataract surgery.
8.12 The Composition for use 4 or any of 8.1 to 8.11, wherein the ocular surface nerves are selected from nerves at the surface of the cornea and/or the conjunctiva
8.13 The Composition for use 4 or any of 8.1 to 8.12, wherein the one or more symptoms associated with ocular surface nerve sensation are selected from eyes feeling gritty, eyes that are sensitive to light (photophobia), painful or sore eyes
8.14 The Composition for use 4 or any of 8.1 to 8.13, wherein the patient is characterized by one or more symptoms selected from
   i. eyes feeling gritty, eyes that are sensitive to light (photophobia) and/or painful or sore eyes, and
   ii. total corneal fluorescein staining (NEI scale) between 5 and 9
8.15 Composition for use 4 or any of 8.1 to 8.14, wherein the patient is a female
8.16 Composition for use 4 or any of 8.1 to 8.14, wherein the patient is a male
8.17 Composition for use 4 or any of 8.1 to 8.16, wherein the patient is aged 20-80 years old at the time of treatment, e.g., 20-50 years old, or 20-70 years old, or 30-80 years old, or 30-50 years old, or 30-70 years old, or 40-80 years old, or 40-60 years old, or 40-70 years old, or 50-80 years old, or 50-70 years old.
8.18 Composition for use 4 or any of 8.1 to 8.17, wherein the patient suffers from a co-morbidity, for example, conjunctivitis, stye, chalazion, blepharitis, ectropion, eyelid laxity, eyelid edema, eyelid dermatitis, punctate keratopathy, or ocular allergies, or any combination thereof.
8.19 Composition for use 4 or any of 8.1 to 8.18, wherein the patient suffers from keratoconjunctivitis sicca which is caused by treatment of a co-morbidity, for example, treatment with any one or more of: isotretinoin, sedatives, diuretics, tricyclic antidepressants, antihypertensives, anticholinergics, oral contraceptives, antihistamine, nasal decongestants, beta-adrenergic antagonists, phenothiazines, atropine opiates (e.g., morphine), optionally wherein any such treatment is concurrent or previous, and further optionally, wherein any such treatment is systemic (e.g., oral or parenteral).
8.20 Composition for use 4 or any of 8.1 to 8.19, wherein the patient suffers from keratoconjunctivitis sicca which is caused by ocular surgical intervention, for example, corneal surgery, refractive surgery, LASIK surgery, cataract surgery, optionally wherein any such ocular surgery is concurrent or previous.
8.21 Composition for use 4 or any of 8.1 to 8.20, wherein the patient is concomitantly under treatment with another topical ophthalmic medication, for example, an antibiotic, antifungal, corticosteroid, immunosuppressant, sympathomimetic, anesthetic, antihistamine, or any combination thereof.
8.22 Composition for use 4 or any of 8.1 to 8.21, wherein the patient is a contact lens wearer.
8.23 Composition for use 4 or any of 8.1 to 8.22, wherein the patient was unresponsive or insufficiently response to previous treatment for keratoconjunctivitis sicca (dry eye disease).
8.24 Composition for use 8.23, wherein said previous treatment comprise one or more of the following treatment methods: topical aqueous immunosuppressant administration (e.g., topical aqueous ciclosporin), topical corticosteroid administration, or topical aqueous artificial tears administration.

Keratoconjunctivitis sicca is a complex, multifaceted disease or condition as described above. It is also known as dry eye syndrome, dry eye disease (DED), or dysfunctional tear syndrome. Aqueous-deficient DED, evaporative DED are within the scope of keratoconjunctivitis sicca and form specific subtypes thereof. Sjögren syndrome, lacrimal gland insufficiency, Meibomian gland disease and Meibomian gland dysfunction, and other conditions are also within the scope of keratoconjunctivitis sicca, being direct or indirect causes thereof.

Meibomian gland diseases cover a broad range of Meibomian gland disorders including neoplasia and congenital disorders. Meibomian gland dysfunction, on the other hand is understood to be abnormalities of the Meibomian glands which are often characterized by gland duct obstructions and/or changes (qualitative and/or quantitative) to the secretions of the glands. In general, conditions or disease states causing or leading to an abnormal, reduced or increased delivery of lipids to the tear film can give rise to keratoconjunctivitis sicca and the symptoms associated therewith.

Symptoms of keratoconjunctivitis sicca include a dry, scratchy, gritty, or sandy feeling in the eye; foreign body sensation; pain or soreness; stinging or burning; itching; increased blinking; eye fatigue; photophobia; blurry vision; redness; mucus discharge; contact lens intolerance; excessive reflex tearing. In addition to the symptoms of keratoconjunctivitis sicca as described, patients with Meibomian gland dysfunction may also experience symptoms including itchiness, redness, swelling, pain or soreness, discharge accumulation or crusting specifically at the lid margins. It is understood that not all patients suffering from keratoconjunctivitis sicca exhibit all symptoms simultaneously. Hence, there is currently no uniform set of criteria for diagnosing the disease. It is also understood that patients may suffer from one or more subtypes of keratoconjunctivitis sicca, or one or more conditions or disease pathways causing keratoconjunctivitis sicca. It is however important to note that, within the scope of the present disclosure, any of the aspects, symptoms or pathophysiological consequences of dry eye disease may be addressed.

Preferably, the patient to be treated with the methods and/or the compositions for use according to the present disclosure is a human patient.

According to a preferred embodiment, the patient to be treated with the methods and/or the compositions for use according to the present disclosure suffers from evaporative dry eye disease (keratoconjunctivitis sicca) associated with Meibomian Gland Dysfunction.

Semifluorinated alkanes are linear or branched alkanes some of whose hydrogen atoms have been replaced by fluorine. The semifluorinated alkanes (SFAs) used in the present disclosure are composed of at least one non-fluorinated hydrocarbon segment and at least one perfluorinated hydrocarbon segment and are according to the general formula F(CF₂)ₙ(CH₂)ₘH. Another nomenclature which may be used herein refers to the above-mentioned SFAs having two segments as RFRH, wherein RF designates a perfluorinated hydrocarbon segment, RH designates a non-fluorinated segment. Alternatively, the compounds may be referred to as FnHm, wherein F means a perfluorinated hydrocarbon segment, H means a non-fluorinated segment, and n, and m is the number of carbon atoms of the respective segment. For example, F6H8 is used for 1-perfluorohexyloctane. Moreover, this type of nomenclature is usually used for compounds having linear segments. Therefore, unless otherwise indicated, it should be assumed that F3H3 means 1-perfluoropropylpropane, rather than 2-perfluoropropylpropane, 1-perfluoroisopropylpropane or 2-perfluoroisopropylpropane.

In some embodiments, the compositions comprising a semifluorinated alkane, as defined in the context of the present disclosure are free of active ingredient, or are drug-free compositions, i.e. free of any pharmaceutically active drug substance useful for ophthalmic treatment. In particular embodiments, the compositions are free of, or exclude a therapeutically effective amount of any active ingredient, or pharmaceutically active drug substance, that is, for example, useful for ophthalmic treatment. As used herein, active ingredient refers to any type of pharmaceutically active compound or derivative that is useful in the prevention, diagnosis, stabilization, treatment, or, generally speaking, management of a condition or disease. Therapeutically effective amount refers to a dose, concentration or strength which is useful for producing a desired pharmacological effect. As used herein, a composition according to the present disclosure which is “free of an active ingredient”, or is “free of a drug substance”, or “free of any pharmaceutically active drug substance useful for ophthalmic treatment,” or similar variations thereof, is a composition which comprises at least one or more semifluorinated alkanes, but does not include any other pharmaceutically active ingredient or drug substance which, e.g. may be useful or active for ophthalmic treatments.

In certain jurisdictions 1-perfluorohexyloctane may be considered as an active pharmaceutical ingredient. Hence, a composition according to the present disclosure which is “free of an active ingredient”, or is “free of a drug substance”, or “free of any pharmaceutically active drug substance useful for ophthalmic treatment,” or similar variations thereof, is a composition which comprises at least 1-perfluorohexyloctane and optionally 2-perfluorohexyloctane, but does not include any other pharmaceutically active ingredient or drug substance which, e.g. may be useful or active for ophthalmic treatments. In other words, besides 1-perfluorohexyloctane and optionally, 2-perfluorohexyloctane, the composition according to the present disclosure does not comprise any active pharmaceutical ingredient.

The SFAs of the disclosure are 1-perfluorohexyloctane (F6H8) and optionally 2-perfluorohexyloctane, in particular embodiments the SFA is 1-perfluorohexyloctane (F(CF₂)₆(CH₂)₃H; F6H8).

In some embodiments, the composition may further comprise a second SFA, namely 2-perfluorohexyloctane (F(CF₂)₆(CH (CH3)) (CH₂)₆H).

Liquid SFAs are chemically and physiologically inert, colourless and stable. Their typical densities range from 1.1 to 1.7 g/cm³ (e.g. the density of F6H8 is 1.35 g/cm³), and their surface tension may be as low as 19 mN/m. SFAs of the F(CF₂)ₙ(CH₂)ₘH type are insoluble in water but also somewhat amphiphilic, with increasing lipophilicity correlating with an increasing size of the non-fluorinated segment.

Liquid SFAs of the RFRH type are being used commercially for unfolding and reapplying a retina, for long-term tamponade as vitreous humour substitute (H. Meinert et al., European Journal of Ophthalmology, Vol. 10(3), pp. 189-197, 2000), and as wash-out solutions for residual silicon oil after vitreo-retinal surgery. Experimentally, they have also been used as blood substitutes (H. Meinert et al., Biomaterials, Artificial Cells, and Immobilization Biotechnology, Vol. 21(5), pp. 583-95, 1993). These applications have established SFA's as physiologically well tolerated compounds.

SFAs are well-tolerated by the eye, as shown in preclinical testing. In comparison, organic or non-aqueous solvents, perhaps with the exception of oily compounds, are typically very irritating or even highly damaging when administered topically to an eye. Moreover, compared to oily carriers or vehicles in ophthalmic compositions for topical use, SFAs exhibit a refractive index in the region of 1.29 to 1.35, which is much better compatible with the aim of a minimally affected vision thus causing little or no blurring. SFA compositions of the present disclosure have several useful functional effects when administered to the eye. Semifluorinated alkanes are able to mix and/or dissolve well with non-polar and lipophilic substances. It is proposed that the SFAs as defined in the context of the present disclosure, e.g. F(CF₂)₆(CH₂)₈H (F6H8), and F(CF₂)₆(CH(CH₃)) (CH₂)₆H (2-perfluorohexyloctane), may be particularly useful for solubilizing meibum lipids and for removing abnormal and obstructive meibum found in clogged Meibomian gland ducts.

Meibum is the lipid secretion of the Meibomian gland ducts and is normally secreted as a clear fluid comprising a complex mixture of polar and non-polar lipids such as cholesterol and wax esters, acyl glycerides, free fatty acids and phospholipids. In their dysfunctional state, the glands producing meibum may express secretions with an altered composition of those lipids which exhibit increased viscosity and which may also contain particulate cellular material. Such secretions can obstruct the gland ducts and may be ineffective for forming a functional stable and continuous tear film lipid layer, leading to lipid tear film deficiency, and the condition and symptoms of keratoconjunctivitis sicca. Ophthalmic compositions comprising a semifluorinated of the formula F(CF₂)ₙ(CH₂)ₘH, as defined in the context of the present disclosure are effective in solubilizing meibum, and in particular, in solubilizing the abnormal (e.g., viscous) meibum obstructing the Meibomian glands and/or Meibomian gland ducts. In addition, the ophthalmic compositions of the present disclosure can also serve as either a replacement, substitute or supplement to the tear film lipid layer. For patients suffering from dry eye syndrome, the SFA compositions of the present disclosure may have a lubricating as well as a protective effect. It is believed that the SFA compositions are capable of forming a protective film over the corneal surface and prevent aqueous evaporative loss of the tear film.

In one embodiment, the ophthalmic SFA compositions as defined in the present disclosure may serve as a replacement, substitute or supplement to the tear film lipid layer, e.g. as a lubricant and/or form a protective film, and also effective for effective in solubilizing meibum, and in particular, in solubilizing the abnormal (e.g., viscous) meibum obstructing the Meibomian glands and/or Meibomian gland ducts

Moreover, SFAs exhibit a remarkable wetting and spreading behaviour by which they can rapidly and effectively spread over the corneal surface and conjunctiva. This remarkable wetting and spreading behaviour permits the SFA to spread away from the administered eye drop rapidly and completely, further permitting the SFA to access the Meibomian gland ducts on the upper and/or lower eyelids. The SFA, due to its high solubilizing capacity, can penetrate the meibum plugs which are prevalent in Meibomian gland dysfunction (MGD) or disease, resulting in solubilization and removal of the plugs, restoring proper Meibomian gland function.

Wetting means the ability of a liquid to establish and maintain contact with a solid surface, resulting from intermolecular interactions when the two are brought together. The balance between adhesive and cohesive forces determines the degree of wetting. The higher the adhesive forces compared to the cohesive forces, the more a drop of liquid will spread across the surface of the solid material. Conversely, very high cohesive forces within the liquid will cause the drop to form a sphere, thus avoiding contact with the surface. Similarly, spreading may also occur at the interface of two liquids which are brought into contact with each other.

A measure for wetting and spreading is the contact angle θ. The contact angle is the angle at which the liquid-vapour interface meets the solid-liquid or liquid-liquid interface. The tendency of a drop to spread out increases as the contact angle decreases. Thus, the contact angle provides an inverse measure of wettability.

A low contact angle of less than 90° indicates high wettability and/or spreading, whereas a higher contact angle indicates poor wettability and spreading. Perfect wetting and spreading results in a contact angle of 0°, also reported as no measurable contact angle.

The enhanced spreading behavior and stable film properties of such ophthalmic compositions comprising SFAs are particularly advantageous for treating the dry eye condition. A droplet administered to the surface of the eye may lead to rapid spreading of the SFA mixture compositions over the corneal surface and the formation of a film. A stable film that does not immediately break up provides a longer-lasting lubricating effect on the ocular surface. Efficient spreading allows for a more effective distribution of the SFA not only over the ocular surface, but also to more distant ocular tissues such as the Meibomian glands or the lacrimal glands.

One result of this is a significantly reduced reliance placed on the blinking mechanism of the patient (which may be ineffective or hindered by the diseased state) to spread the composition over the ocular surface. It is believed that the compositions of the present disclosure may thus be more efficiently administered to the ocular surface, in comparison with conventional formulations which are generally aqueous based and have poorer spreading behavior. As such, less frequent administration to the dry eye for relief may be achieved with these compositions.

In particular, the compositions of the present disclosure as described in the above embodiments may be used for the treatment of patients who are non-responsive to traditional physical methods of treating Meibomian gland dysfunction, or dry eye disease caused, or exacerbated by Meibomian gland dysfunction, such as physical or forced expression of meibum or meibum obstructions from the Meibomian glands, application of heat compresses, e.g. to the eyelids (heat therapy), simultaneous physical expression and heat therapy, lid scrubs, or intraductal probing of the meibomian gland orifices. Non-responsive to treatment may refer to a continued condition of, a progression, or a recurrence of meibomian gland dysfunction and symptoms associated thereof in a patient, despite a prescribed or recommended period of treatment, e.g. using the traditional methods of treatment. The use of the present compositions and methods of treatments according to the disclosure may be used to replace such therapy, or also as an alternative therapy to such traditional methods, which often may need to be performed at a doctor's office and which are not as convenient and/or poorly tolerated due to pain during the application of these physical methods.

In another aspect, the compositions for the present disclosure may be used for the treatment of conditions such described in the above embodiments, wherein the patient is non-responsive to treatment with aqueous ophthalmic eye drop compositions. In particular, the compositions may be used for the treatment of patients suffering from meibomian gland dysfunction and who are non-responsive to treatment with aqueous-based ophthalmic eye drop compositions e.g. emulsions, or aqueous solutions such as tear supplements or tear substitutes, and who may still have a continuing condition of, a progression of or a recurrence of dry eye disease or MGD, or symptoms thereof, despite a course of therapy with such compositions.

Another advantage of using ophthalmic compositions comprising SFA is that SFAs are capable of forming very small droplets, for example, of about 10-12 μl, or 10-11 μl or 11 μl volume, when dispensed from a conventional dropper such as a conventional eye dropper. Without wishing to be bound by theory, it is believed that the small droplet size is a result of an interplay of the SFA's unique properties in terms of their density, viscosity, and surface tension. It is believed that for topical administration into an eye a small drop or volume of administration is highly advantageous as the capability of the lacrimal sac to accept and hold fluid is extremely limited. In fact, it is very common that the administration of a conventional eye drop formulation based on water or oil immediately leads to a discharge of a substantial fraction of the administered medicine as well as some tear fluid. At the same time, there is a risk that some of the administered dose will be taken up systemically via the nasolacrimal duct.

The present disclosure also provides a means of formulating non-aqueous ophthalmic compositions which are microbiologically stable. Aqueous ophthalmic compositions are prone to bacterial contamination. In comparison, SFAs have bacteriostatic properties and do not support microbial growth. Hence, it is possible to formulate preservative-free ophthalmic compositions which are better tolerable for many patients, in particular patients suffering from keratoconjunctivitis sicca. Such compositions also do not promote bacterial infection of the eye lid margin in patients who, for example, are suffering from obstructed or blocked Meibomian glands.

Ophthalmic tissue includes any surface of the eye anatomy that is, or can be (i.e. by non-surgical means) topically exposed. Optionally, the compositions are administered as a single drop to either the cornea or conjunctiva. Ophthalmic tissue includes, but is not limited to, cornea, conjunctiva (bulbar and palpebral), lacrimal glands (including lacrimal ducts and lacrimal sacs), the Meibomian glands, and the sclera.

In some embodiments, the compositions of the present disclosure can be used to alleviate or relieve ocular symptoms associated ophthalmic disorders or conditions, including keratoconjunctivitis sicca and Meibomian gland dysfunction. For example, they may be used in addition to medicines comprising an active ingredient whose dosing frequency is typically limited by tolerability or safety concerns. The compositions for alleviating or relieving any non-disease related sensation of dryness, irritation, or discomfort of the eye. Said compositions may be used concomitantly or in conjunction with eye compositions with pharmaceutically active ingredients (e.g. immunosuppressant eye drops) that are aimed at curing or treating the root causative pathways of an ophthalmic disease.

In some embodiments, the compositions of the present disclosure may be used as a cleansing solution for the eye or ophthalmic tissue. The compositions are used to cleanse or help remove or wash away any accumulated debris or discharge such as meibum secretions from the eye lid, eye lid margins, eye lashes, or eye crevices. Compared to aqueous formulations, the SFA compositions are able to spread more readily, and thus are able to reach the more difficult to access regions of eye lid anatomy. In a particular embodiment, the compositions for use as a cleansing solution are formulated to be administered as a spray. This can be useful for patients either averse to, or unable to apply the compositions via eye drops.

Optionally the compositions of the present disclosure are highly stable, water-free, preservative-free.

All patents, publications, and other references described herein are hereby incorporated by reference in their entireties.

### EXAMPLES

### Example 1: Clinical Study US

A Phase 2, Multi-Center, Randomized, Double-Masked, Saline-Controlled Study to Evaluate the Effect of 1-Perfluorohexyloctane (NOV03) at two different dosing regimens on signs and symptoms of Dry Eye Disease (DED) was conducted. The study was performed at 11 investigational cites in the United States. The study was reviewed and approved by the respective ethics committees and registered at www.clinicaltrials.gov (NCT03333057).

The primary objective for this study is to evaluate the efficacy, safety, and tolerability of an ophthalmic composition essentially consisting of 1-perfluorohexyloctane (NOV03) at two different dosing regimens (QID, BID) compared to saline solution in subjects with Dry Eye Disease. The secondary objectives are to compare the effect of an ophthalmic composition essentially consisting of 1-perfluorohexyloctane (NOV03) and saline solution at two different dosing regimens on signs and symptoms of Dry Eye Disease and to evaluate the pharmacokinetics after 57 days dosing.

### Inclusion Criteria:

Subjects must:
a. Be at least 18 years of age.
b. Provide written informed consent.
c. Have a subject reported history of Dry Eye Disease in both eyes for at least 6 months prior to Visit 0.
d. Have Tear film break-up time (TFBUT)≤5 sec at Visit 0 and Visit 1.
e. Have Ocular Surface Disease Index (OSDI)≥25 at Visit 0 and Visit 1.
f. Have a Schirmer's Test I≥5 mm at Visit 0 and Visit 1.
g. Have Meibomian Gland Dysfunction (MGD) defined as MGD score≥3 (secretion of 5 central glands on lower eyelid will be evaluated, each will be scored from 0-3; 0=normal, 1=thick/yellow, whitish, particulate 2=paste; 3=none/occluded; total score will range from 0-15) at Visit 0 and Visit 1.
h. Have a total corneal fluorescein staining score of 4≤X≤11 (i.e. sum of inferior, superior, central, nasal, and temporal) according to the National Eye Institute (NEI) grading at Visit 0 and Visit 1.
i. Have at least one eye (the same eye) satisfy all criteria for d, f, g, and h above at Visit 0 and Visit 1.
j. Be able and willing to follow instructions, including participation in all study assessments and visits.

### Exclusion Criteria: (Excerpt)

Subjects must not:
a. Women who are pregnant, nursing or planning pregnancy
b. Unwillingness to submit a blood pregnancy test at screening and the last visit (or early termination visit) if of childbearing potential, or unwillingness to use acceptable means of birth control
c. Clinically significant slit-lamp findings or abnormal lid anatomy at screening
d. Ocular/peri-ocular malignancy
e. History of herpetic keratitis
f. Active ocular allergies or ocular allergies that are expected to be active during the study
g. Ongoing ocular or systemic infection
h. Wear contact lenses within 1 month prior to screening or anticipated use of contact lenses during the study
i. Intra-ocular surgery or ocular laser surgery within the previous 6 months, or have planned ocular and/or lid surgeries over the study period
j. Presence of uncontrolled systemic diseases
k. Presence of known allergy and/or sensitivity to the study drug or saline components
l. Use of any topical steroids treatments, topical cyclosporine, lifitegrast, serum tears or topical anti-glaucoma medication within 2 months prior to screening

Subjects eligible to be randomized, received one of the following treatments to be administered bilaterally from Visit 1 to Visit 4:

| | |
|---|---|
| Treatment 1: NOV03 (ophthalmic composition essentially | 4 times daily |
| consisting of 1-perfluorohexyloctane); VERUM | (QID) |
| Treatment 2: NOV03 (ophthalmic composition essentially | 2 times daily |
| consisting of 1-perfluorohexyloctane); VERUM | (BID) |
| Treatment 3: Saline solution (0.9% sodium chloride | 4 times daily |
| solution); PLACEBO | (QID) |
| Treatment 4: Saline solution (0.9% sodium chloride | 2 times daily |
| solution); PLACEBO | (BID) |

After being trained on how to use the treatments, patients were advised to apply 1 drop of the respective treatment in each of both eyes, either 4 times or respectively 2 times daily.

The drop volume of a single drop of NOV03 (ophthalmic composition essentially consisting of 1-perfluorohexyloctane; d=1.35 g/ml) relates to 10-12 μl, translating to 13.5-16.2 mg for a single dose per eye or to a daily dose of 27-32.4 mg (20-24 μl) per eye for a 2 times daily treatment (BID) or respectively to a daily dose of 54-64.8 mg (40-48 μl) per eye for a 4 times daily treatment (QID).

The drop volume of a single drop of the Saline solution (0.9% sodium chloride solution) relates to 35-40 μl, translating to a daily dose of 70-80 μl per eye for a 2 times daily treatment (BID) or respectively to a daily dose of 140-160 μl per eye for a 4 times daily treatment (QID).

In the following the NOV03 (ophthalmic composition essentially consisting of 1-perfluorohexyloctane) treatment is also referred to a “Verum”, while the Saline (0.9% sodium chloride solution) treatment is also referred to as “Placebo”.

### Visit Schedule:

This study will consist of two periods: a 14-day screening period and a 57-day treatment period.

Screening (Visit 0); Within 14 days before Visit 1 Subjects will be required to sign an Informed Consent before completing any study related procedure. At the screening visit, vital signs will be assessed and the subject will give blood for safety laboratory tests. They will also submit to a battery of tests to confirm the extent and severity of their symptoms and objective signs of dry eye. At least one eye must qualify with the following objective measures: Tear film break up time sec, Schirmer's Test mm, and Meibomian gland dysfunction (MGD) defined as MGD score 3 inclusive.

Baseline Visit Day 1 (Visit 1); On Day 1 (Visit 1), eligible subjects will be evaluated for baseline signs and symptoms of dry eye disease. After randomization subjects at selected sites will give a blood sample to be used for PK. Subjects will be given a 14-day supply and will self-administer a single drop of the study medication into each eye at the clinic. Each subject will be given a diary to record that their doses were taken. Study staff will help the subject to understand how to use the diary and when the remaining doses should be taken.

Visits 2-4; Subjects will return to the clinic on Day 15±1 (Visit 2), 29±2 (Visit 3), and 57±2 (Visit 4) to be evaluated for signs and symptoms of dry eye disease. During this period, subjects will dose NOV03 or the saline solution QID and BID, depending on their assigned group. The unused portion of the study medication should be returned to the clinic and a new study medication kit will be dispensed. The diary will be checked. At Visit 4, vital signs will be evaluated and a second blood draw will be performed for PK at selected sites. The diary will be collected at the clinic during each visit. Subjects will be dismissed from the study after all Visit 4 assessments have been completed.

### Patients and Examination Parameters

336 patients meeting the inclusion/exclusion criteria were selected by the investigational sites. The study population represents a highly symptomatic dry eye disease (DED) population with significant MGD involvement as evidenced at baseline by low TBUT (mean TBUT˜3), high OSDI score (mean OSDI˜55), high VAS severity of dryness score (mean VAS severity of dryness score˜69) and high MGD Score (mean MGD score˜7.6).

Parameters determined both at the baseline visit and the following visit included OSDI Questionnaire, 10-item Visual Analog Scale (VAS) Questionnaire, Visual Acuity (ETDRS), Slit-lamp Biomicroscopy, TFBUT, Fluorescein Staining NEI grading, Lissamine Green Staining Oxford scale, Meibomian Gland Assessment (MGD score), Schirmer's Test I (without anesthesia).

323 patients completed the study, with 110 patients in NOV03/QID, 105 patients in the NOV03/BID and 108 patients in the Saline/QID+BID arm. Statistical analysis of the examination parameters was conducted to identify statistically significant differences between the verum and the placebo arms.

(a) Corneal Fluorescein Staining

For staining 5 μL of 2% preservative-free sodium fluorescein solution are instilled into the inferior conjunctival cul-de-sac of each eye (a fluorescein strip might be used but only at Visit 0) In order to achieve maximum fluorescence, it was waited for approximately 2-3 minutes after instillation before evaluating fluorescein staining. A Wratten #12 yellow filter will be used to enhance the ability to grade fluorescein staining. The staining will be graded with the NEI (National Eye Institute) Grading Scale. Only the staining of the cornea will be graded. Digital images of fluorescein staining may be taken for digital analysis.

Based on the NEI/Industry Workshop Scale, the grade of the ocular surface damage for each eye is scored for each of the five regions of the cornea based on measuring fluorescein uptake. In the NEI/Industry Workshop scale, the cornea of the right eye (commonly denoted as OD) and cornea of the left eye (commonly denoted as OS) are each assessed by diagrammatically as an approximate circular area divided into 5 regions comprising of: a central circular area representative as the central corneal region (region 1), with the remaining circumferential area divided into four quadrants representing the superior corneal region (region 2), inferior corneal region (region 5) as the upper and lower quadrants respectively, and the nasal corneal region (region 4, located adjacent relative to a subject's nose) and temporal corneal region (region 3, adjacent relative to a subject's temples) representing the side quadrants.

According to the NEI Grading Scale a standardized grading system of 0-3 is used to define the surface damage for each of these five regions on each cornea (central, superior, temporal, nasal, inferior). Grade 0 will be specified when no fluorescein staining is present. Grades 1, 2 and 3 define an increasing density and degree of observed fluorescein staining, The maximum total score for each eye is 15.

(b) Ocular Surface and Disease Index (OSDI)© Questionnaire

The Ocular Surface Disease Index (OSDI©) is perhaps the most frequently used survey instrument for the assessment of ocular surface disease severity in dry eye research. The OSDI© was created by the Outcomes Research Group at Allergan Inc in order to quickly assess the symptoms of ocular irritation in dry eye disease and how they affect functioning related to vision. This 12-item questionnaire assesses dry eye symptoms and the effects it has on vision-related function in the past week of the patient's life. The questionnaire has 3 subscales: ocular symptoms, vision-related function, and environmental triggers. Patients rate their responses on a 0 to 4 scale with 0 corresponding to “none of the time” and 4 corresponding to “all of the time.” A final score is calculated which ranges from 0 to 100.

The questions assess the following: dry eye symptoms experienced by the patient within past week: sensitivity to light, gritty sensation, pain or sore eyes, blurriness, and poor vision; vision-related function, in terms of problems in the past week with regards to: reading, driving at night, working on a computer or bank machine, watching television; and in terms of environmental factors or triggers i.e. discomfort experienced in the past week during: windy conditions, places with low humidity, and areas with air condition.

Subtotals are obtained for all the questions, as well as the total number of questions answered. The OSDI index is assessed based on a scale of 0 to 100, with higher scores representing a greater disability. The OSDI index is calculated from the sum of the scores multiplied by a factor of 25, over the total number of questions answered. Higher scores represent a greater degree/severity and impact of dry eye disease.

(c) Visual Analog Scale (VAS); Eye Dryness Score

An Eye Dryness Score was determined based on a 10-item questionnaire provided to subjects, where subjects were asked to rate their ocular symptoms (both eyes simultaneously) due to ocular dryness by placing a vertical mark on a horizontal line scale to indicate the level of discomfort (0% corresponds to “no dryness” and 100% corresponds to “maximal dryness”). Subjects are asked about the severity of dry eye symptoms, namely the symptoms of dryness representing the first 8 questions of the VAS questionnaire corresponding to: dryness (corresponding to the first question in the VAS questionnaire, and also referred to in the text and in the graphs as “severity of dryness”; with the terms “dryness” and “severity of dryness” being used interchangeably herein as a dry eye symptom designation, sticky feeling, burning/stinging, foreign body sensation, itching, blurred vision, sensitivity to light, and pain.

Subjects are also asked about their awareness of their dry eye symptoms and frequency of their dry eye symptoms, namely questions 9-10 of the VAS questionnaire. The rating for these questions is indicated by the subject by placing a vertical mark on a horizontal line scale to indicate the percentage of time of awareness or frequency, with 0% corresponding to ‘never’ and 100% corresponding to ‘all of the time’.

The assessment line length of the scale for all questions is 100 mm (10 cm), with grading provided at every 10 mm (suggesting 10%, 20%, etc).

(d) Tear Film Break-Up Time (TFBUT)

For analysis 5 μL of 2% preservative-free sodium fluorescein solution are instilled into the inferior conjunctival cul-de-sac of each eye (a fluorescein strip might be used but only at Visit 0). To thoroughly mix the fluorescein with the tear film, the subject was be instructed to blink several times. In order to achieve maximum fluorescence, it was waited for approximately 30 seconds after instillation before evaluating TFBUT.

With the aid of a slit-lamp, the integrity of the tear film was monitored, noting the time it takes to form micelles from the time that the eye is opened. TFBUT will be measured in seconds using a stopwatch and a digital image recording system for the right eye followed by the left eye. A Wratten #12 yellow filter was used to enhance the ability to grade TFBUT.

(e) Meibomian Gland Assessment (MGD Score)

Meibomian gland dysfunction (MGD) is a blockage or some other abnormality of the meibomian glands so they don't secrete enough oil into the tears. Because the tears then evaporate too quickly, MGD is a leading cause of dry eye syndrome.

For analysis of the Meibum, the Meibomian Gland Evaluator stick (Korb MGE®-Stick; Tear Science, Morrisville, US) allowing for a reproducible and a standardized force application (1.25 g/mm2). The MGE-stick was used according to the instructions of the manufacturer.

For analysis, the secretion (Meibum) of 5 central Meibomian glands on the lower eyelid was obtained by expressing the glands by standardized force of 1.25 g/mm2 utilizing the MGE-stick and evaluated. The expressed secretion (Meibum) was scored on a scale from 0 to 3, with 0=normal, 1=thick/yellow, whitish, particulate; 2=paste; 3=none/occluded. Therefore, the MGD-score represents the sum of the scores of the 5 central Meibomian Glands, thus the total score will range from 0-15.

Herein, a MGD score of equal or higher than 6 relates to at least 3 out of 5 central meibomian glands presenting as pasty (thick) matter or being occluded upon expressing the meibum from said glands by a standardized force of a but 1.0-2.0 g/mm2, preferably by a standardized force of about 1.25 g/mm2.

Further, a MGD score of equal or higher than 7 relates to at least 2 out of 5 central meibomian glands presenting as pasty (thick) matter and at least 1 central meibomian glands presenting as being occluded upon expressing the meibum from said glands by a standardized force of a but 1.0-2.0 g/mm2, preferably by a standardized force of about 1.25 g/mm2.

(f) Schirmer I Test

The Schirmer I test (also referred to herein as Schirmer's Test I) is a simple test to assess aqueous tear production. In this test, a strip of filter paper is placed on the lower eyelid margin without anesthesia, after 5 minutes, the strip is removed, and the amount of wetting is measured in millimeters. It is generally agreed that a Schirmer I test of 5 mm or less in 5 min is related to an abnormal tear production (not enough tear fluid being produced). On the other hand, individuals characterized by a Schirmer I test of equal or greater than 10 mm are considered to have normal tear production.

### Results NOV03-Treatment (QID)

The examination parameters were compared between 4 times daily treatment (QID) of NOV03 (ophthalmic composition essentially consisting of 1-perfluorohexyloctane; Verum) with Placebo (Saline solution; 0.9% sodium chloride solution; QID+BID).

The study demonstrated relevant and statistically significant improvements in both signs and symptoms in a highly symptomatic dry eye disease (DED) population with significant MGD involvement when treated 4 times daily by a single drop of 10-12 μl of an ophthalmic composition essentially consisting of 1-perfluorohexyloctane to the eye of patient.

The study met its prespecified primary efficacy endpoint of total corneal fluorescein staining demonstrating the reduction of the ocular surface damage of the total corneal region at 8 weeks for the QID dosing regimen.

Additionally, clear improvements were observed for the reduction of the ocular surface damage also for the central corneal region, the nasal corneal region, the temporal corneal region and the inferior corneal region as evidenced by corresponding fluorescein staining determinations of the central, nasal, temporal, inferior corneal region. Notably, the reduction of the ocular surface damage of the central cornea region is highly important, as the central corneal region is in the center of the visual axis and thus improvement in that respect is directly linked to the visual acuity of the patient. The superior corneal region did not show such clear improvement in respect to ocular surface damage [See FIG. 1(a) to (f)]

The treatment effect relating to the signs started surprisingly early (2 weeks) and was significant throughout the visit (at 4 weeks, 8 weeks).

Furthermore, the study showed highly statistical significant improvement in various symptoms over the placebo group, determined by the Eye Dryness Score on a visual analog scale (VAS), including “awareness of dryness”, “severity of dryness”, “frequency of dryness”, “burning/stinging”, “itching”, “sticky feeling”, “blurred vision”, “foreign body sensation”, “sensitivity to light”. The VAS symptom “pain” did show higher variability and did not improve at the 4 week timepoint. [see FIG. 2(a)-(j)]

Also here, the treatment effect relating to the VAS symptoms started surprisingly early (2 weeks) and was significant throughout the visit (at 4 weeks, 8 weeks).

Further, the study showed highly statistical significant improvement in various symptoms over the placebo group, determined by ocular surface disease index (OSDI) score, including total OSDI score [see FIG. 3].

Additionally, clear improvements were observed for individual symptoms determined by the ocular surface disease index (OSDI) questionnaire for “sensitivity to light”, “eyes feeling gritty”, “painful or sore eyes”, “blurred vision”, “poor vision”, “reading problems”, “problems with driving at night”, “problems with watching TV”, “uncomfortable under windy conditions”, “uncomfortable in areas with low humidity” and “uncomfortable in areas that are air conditions”. The OSDI symptom “problems with working with a computer or bank machine (ATM)” did show higher data variability and did not improve at the 4 and 8-week timepoint. [see FIGS. 4(a)-(l)].

Also here, treatment effect relating to the OSDI symptoms started surprisingly early (2 weeks) and was significant throughout the visit (at 4 weeks, 8 weeks).

It was found that the NOV03-Treatement (QID) was beneficial for patients that are highly symptomatic (i.e. characterized by a high OSDI score) and present with significant MGD involvement (i.e. characterized by a low TBUT, or high MGD score) and/or moderate ocular surface damage of the cornea (i.e. characterized by a moderate total corneal fluorescein staining).

It was further found that the response to the NOV03-treatment (QID) with regard to improvement in corneal staining parameters was starting early and resulted in surprisingly high response rates. Herein, an improvement of grades in total corneal staining was found in 32% of patients after 2 weeks of treatment and in 42% of patients after 8 weeks. Notably, an improvement of grade in central corneal staining was found in 39% of patients after 2 weeks of treatment and in 50% of patients after 8 weeks, which translates to a significant reduction of visual impairment originating from evaporative dry eye disease associated with Meibomian Gland Dysfunction.

The study revealed that the NOV03-Treatment (QID) was beneficial for a population of patients that are highly symptomatic (i.e. characterized by a high OSDI score, i.e. of between 38 and 72) and present with significant MGD involvement (i.e. characterized by a low TBUT, i.e. of between 2.1 and 3.9 seconds, or by a high MGD score, i.e. of between 4.0 and 11.2) and/or moderate ocular surface damage of the cornea (i.e. characterized by a moderate total corneal fluorescein staining, i.e. of between 4.8 and 9.2) [see FIGS. 1-4].

Therefore, patients especially benefitted from the NOV03-Treatment (QID) when meeting at baseline (before starting the therapy) one or more criteria selected from the group consisting of a tear film breakup time (TBUT) of equal or lower than 3 and/or an ocular surface disease index (OSDI) of higher than 57 and/or a total corneal fluorescein staining (NEI scale) between 5 and 9 and/or a MGD score of equal or higher than 7 [see FIG. 5] and/or a Schirmer I Test of greater or equal than 10 mm.

Surprisingly, it was also found that individuals that suffer from dry eye disease associated with Meibomian gland dysfunction (for example having an MGD score of equal to higher than 7) but characterized by relatively normal tear production as indicated by a Schirmer I Test scoring of greater or equal than 10 mm, benefitted from the NOV03-Treatment (QID) [See FIG. 6], in respect to treatment of the symptom of dryness. As described above, the Schirmer I test assesses aqueous tear production and the threshold value of 10 mm or above typically indicates unimpaired, or normal tear production. Unexpectedly, it was observed that a significant change from baseline i.e. improvement with regards to the treatment/alleviation of the dry eye disease symptom of severity of dryness was observed for patients with comparatively normal tear production levels.

### FIGURES

FIG. 1 (Ocular Surface Damage): Improvement of the ocular surface damage of the (a) total corneal region, (b) central corneal region, (c) nasal corneal region, (d) inferior corneal region, (e) temporal corneal region and (f) superior corneal region as determined by fluorescein staining (see experimental section for details on the corneal fluorescein staining and grading according to the NEI scale). Depicted is the change from baseline (Visit 1, Day 1) for Visit (2 weeks), Visit 3 (4 weeks) and Visit 4 (8 weeks), with Verum representing the 4-time daily treatment (QID) with NOV03 (ophthalmic composition essentially consisting of 1-perfluorohexyloctane; solid line) and Placebo representing the Saline solution (0.9% sodium chloride solution; QID+BID; broken line).

FIG. 2 (Symptoms—Visual Analog Scale (VAS)): Improvement of the symptoms of dryness determined by the Eye Dryness Score on a visual analog scale (VAS), including (a)“severity of dryness” (corresponding to question 1 “dryness” of the 10-item VAS questionnaire), (b) “frequency of dryness”, (c) “awareness of dryness”, (d) “burning/stinging”, (e) “itching”, (f) “sticky feeling”, (g) “blurred vision”, (h)“foreign body sensation”, (i) “sensitivity to light”, (j) “pain” (see experimental section for details on Visual Analog Scale (VAS) questionnaire). Depicted is the change from baseline (Visit 1, Day 1) for Visit (2 weeks), Visit 3 (4 weeks) and Visit 4 (8 weeks), with Verum representing the 4-time daily treatment (QID) with NOV03 (ophthalmic composition essentially consisting of 1-perfluorohexyloctane; solid line) and Placebo representing the Saline solution (0.9% sodium chloride solution; QID+BID; broken line).

FIG. 3 (Symptoms—Total Ocular Surface Disease Index (OSDI)): Improvement of the symptoms of dryness determined by the ocular surface disease index (OSDI) score, including total OSDI score (see experimental section for details on Visual Analog Scale (VAS) questionnaire). Depicted is the change from baseline (Visit 1, Day 1) for Visit (2 weeks), Visit 3 (4 weeks) and Visit 4 (8 weeks), with Verum representing the 4-time daily treatment (QID) with NOV03 (ophthalmic composition essentially consisting of 1-perfluorohexyloctane; solid line) and Placebo representing the Saline solution (0.9% sodium chloride solution; QID+BID; broken line).

FIG. 4 (Symptoms—Total Ocular Surface Disease Index (OSDI)): Improvement of individual symptoms of dryness determined by the ocular surface disease index (OSDI) score, including (a) “sensitivity to light”, (b) “eyes feeling gritty”, (c) “painful or sore eyes”, (d) “blurred vision”, (e) “poor vision”, (f) “reading problems”, (g) “problems with driving at night”, (h) “problems with working with a computer or bank machine (ATM)”, (i) “problems with watching TV”, (j) “uncomfortable under windy conditions”, (k)“uncomfortable in areas with low humidity” and (l)“uncomfortable in areas that are air conditions” (see experimental section for details on Visual Analog Scale (VAS) questionnaire). Depicted is the change from baseline (Visit 1, Day 1) for Visit (2 weeks), Visit 3 (4 weeks) and Visit 4 (8 weeks), with Verum representing the 4-time daily treatment (QID) with NOV03 (ophthalmic composition essentially consisting of 1-perfluorohexyloctane; solid line) and Placebo representing the Saline solution (0.9% sodium chloride solution; QID+BID; broken line).

FIG. 5 (Patients especially benefitting from the NOV03-Treatment (QID)): Improvement of the symptoms of dryness determined by the Eye Dryness Score on a visual analog scale (VAS), including: (a)“severity of dryness” (corresponding to question 1 “dryness” of the 10-item VAS questionnaire) and (b) “frequency of dryness” in a subpopulation of patients characterized by a MGD score 7. Improvement of the ocular surface damage of the cornea determined by fluorescein staining and grading of the total corneal region: (c) in a subpopulation of patients characterized by a MGD score 7, (d) in a subpopulation of patients characterized by a TFBUT 3. (see experimental section for details on Visual Analog Scale (VAS) questionnaire, MGD score, TFBUT, fluorescein staining). Depicted is the change from baseline (Visit 1, Day 1) for Visit (2 weeks), Visit 3 (4 weeks) and Visit 4 (8 weeks), with Verum representing the 4-time daily treatment (QID) with NOV03 (ophthalmic composition essentially consisting of 1-perfluorohexyloctane; solid line) and Placebo representing the Saline solution (0.9% sodium chloride solution; QID+BID; broken line).

FIG. 6 (Patients especially benefitting from the NOV03-Treatment (QID)): Improvement of the symptom “severity of dryness” determined by the Eye Dryness Score on a visual analog scale (VAS), in a subpopulation of patients characterized by a Schirmer I Test of equal or greater than 10 mm compared to the general population of patients, after 8 weeks of treatment. Change from baseline is depicted for the NOV03-Treatment (QID) as well as the control saline solution (0.9% sodium chloride solution; QID+BID) The subgroup of patients with greater than 10 mm Schirmer I scores are considered to have a normal tear production, but suffering from dry eye disease associated with Meibomian Gland Dysfunction.

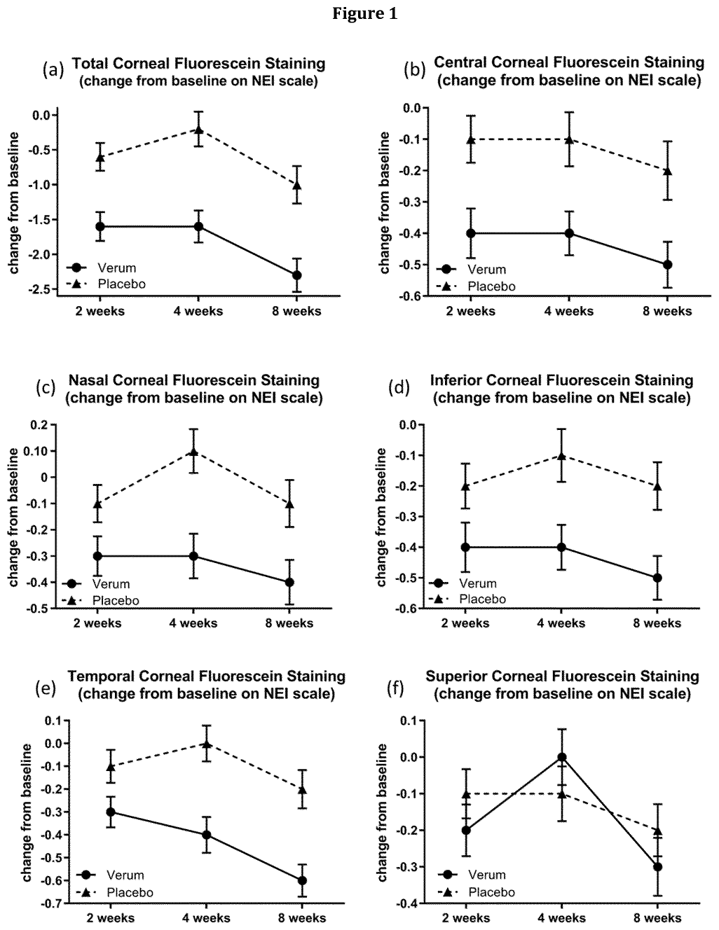

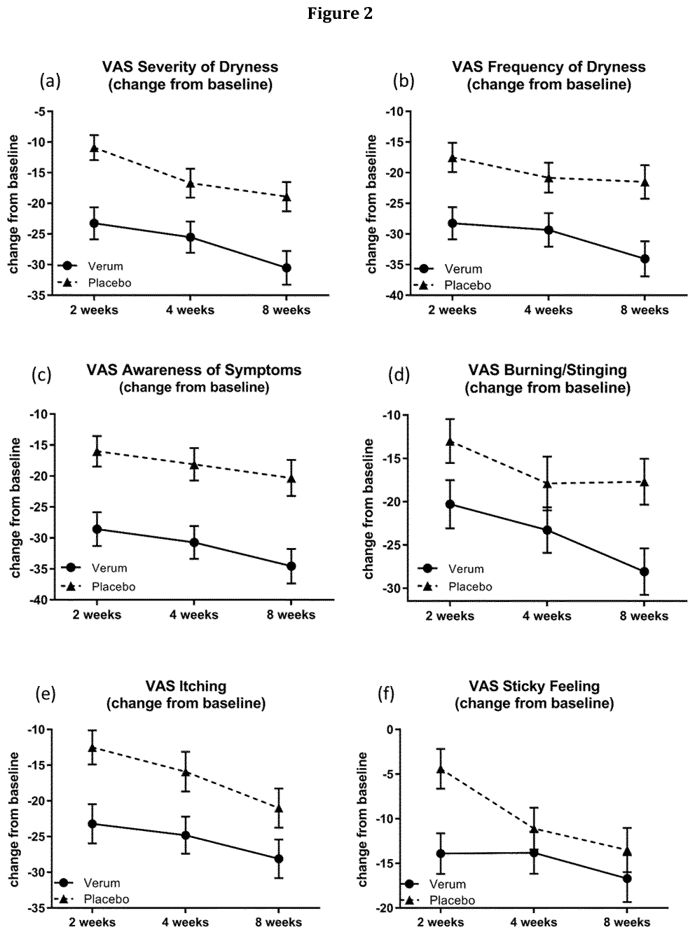

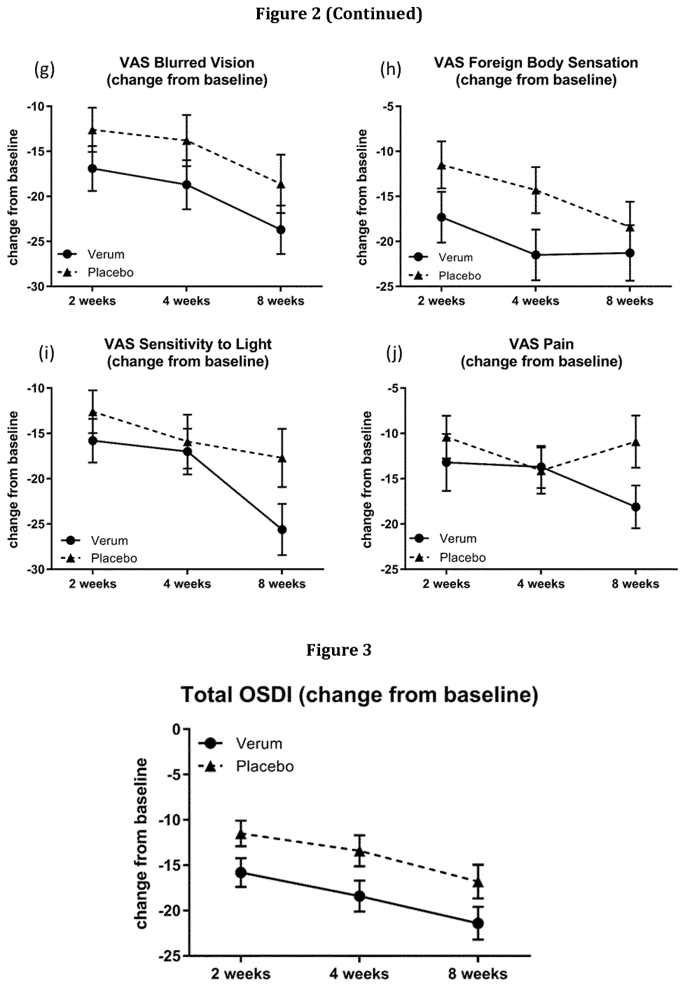

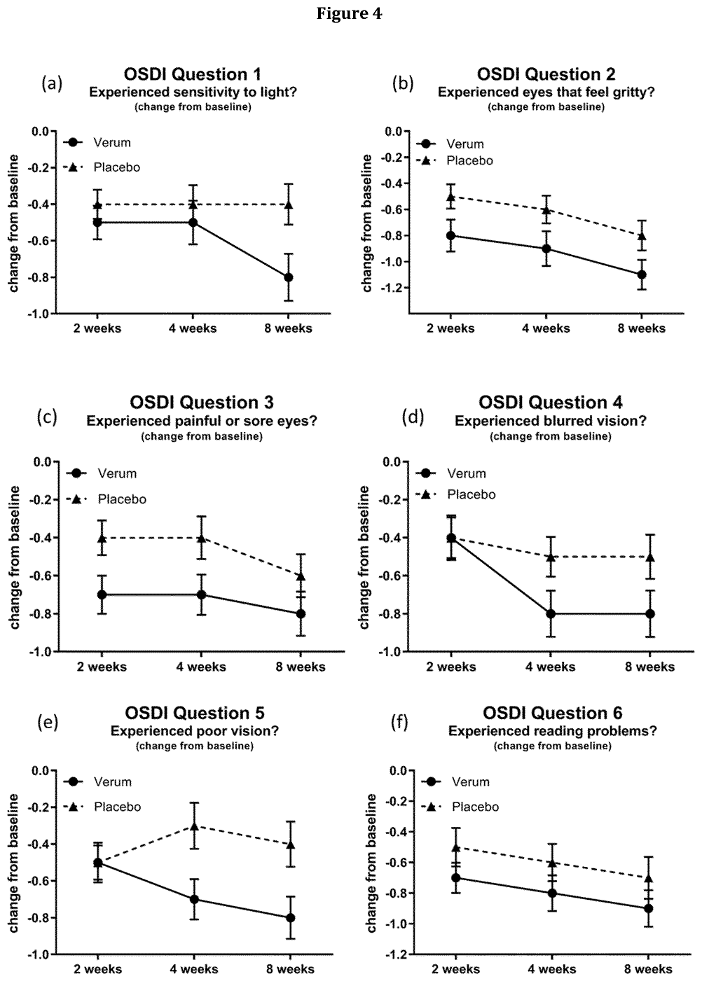

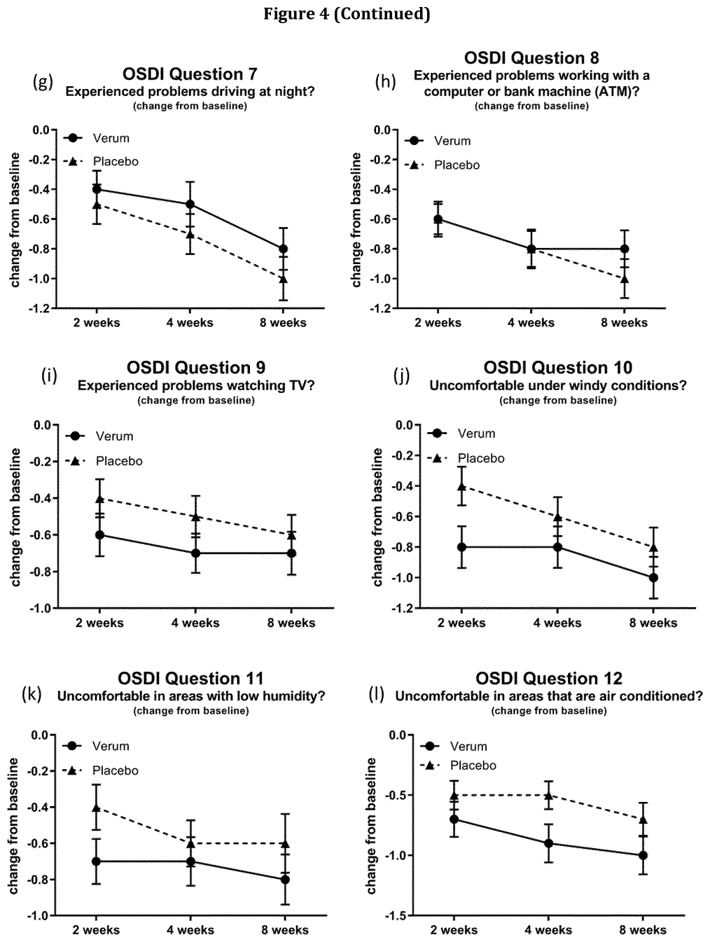

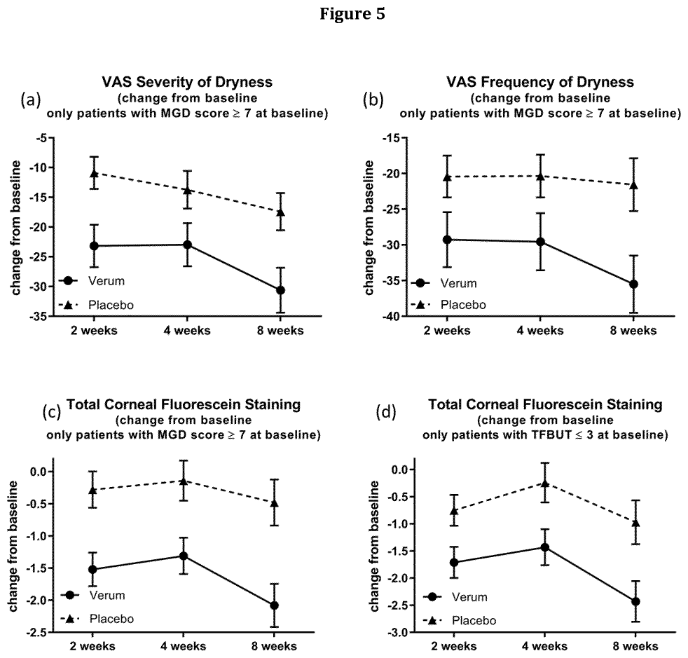

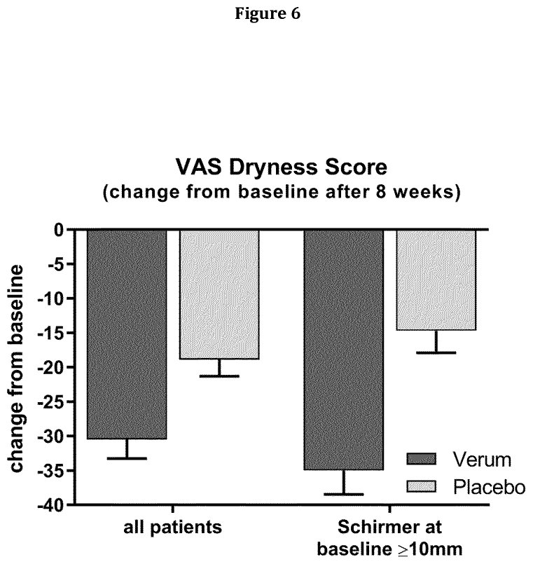

1. A method for the treatment of severity of dryness in a patient suffering from dry eye disease associated with Meibomian Gland Dysfunction, the method comprising administering to a patient in need thereof, a composition which consists essentially of 1-perfluorohexyloctane, and wherein the composition is topically administered for up to 4 times daily as a single drop of about 10-12 μl to the eye of a patient.

2. A method for the treatment of ocular surface damage of the central corneal region, in a patient suffering from dry eye disease associated with Meibomian Gland Dysfunction, the method comprising administering to a patient in need thereof, a composition which consists essentially of 1-perfluorohexyloctane, and wherein the composition is topically administered for up to 4 times daily as a single drop of about 10-12 μl to the eye of a patient.

3. A method for the treatment of ocular surface damage of the central corneal region and for use in the treatment of severity of dryness in a patient suffering from dry eye disease associated with Meibomian Gland Dysfunction, the method comprising administering to a patient in need thereof, a composition which consists essentially of 1-perfluorohexyloctane, and wherein the composition is topically administered for up to 4 times daily as a single drop of about 10-12 μl to the eye of a patient.

4. The method according to claim 1, wherein the composition is administered as a single drop of about 11 μl to the eye of a patient.

5. The method according to claim 1, wherein the composition is administered four times per day to the eye of a patient.

6. The method according to claim 1, wherein the patient to be treated is highly symptomatic with significant involvement of Meibomian Gland Dysfunction.

7. The method according to claim 1, wherein the patient to be treated is characterized by one or more criteria selected from:
(i) a tear film breakup time (TBUT) between 2.1 and 3.9 sec,
(ii) an ocular surface disease index (OSDI) of between 38 and 72,
(iii) a total corneal fluorescein staining (NEI scale) between 4.8 and 9.2,
(iv) a MGD score between 4.0 and 11.2, and
(v) a Schirmer I Test of equal or greater than 5 mm.

8. The method according to claim 1, wherein the patient to be treated is characterized by one or more criteria selected from:
(i) a tear film breakup time (TBUT) of lower than 3 sec,
(ii) an ocular surface disease index (OSDI) of higher than 57,
(iii) a total corneal fluorescein staining (NEI scale) between 5 and 9,
(iv) a MGD score of equal or higher than 7, and
(v) a Schirmer I Test of equal or greater than 10 mm.

9. The method according to claim 1, wherein the composition is further effective in treating (reducing) the ocular surface damage of the total corneal region and/or the nasal corneal region and/or the temporal corneal region and/or the inferior corneal region.

10. The method according to claim 1, wherein the composition is further effective in treating (reducing) the frequency of dryness and/or the awareness of dryness and/or the burning/stinging and/or the itching and/or the sticky feeling and/or the blurred vision and/or the foreign body sensation and/or the total ocular surface disease index (OSDI) score.

11. The method according to claim 1, wherein the patient is not suffering from aqueous-deficient dry eye disease and/or wherein the patient is suffering from evaporative keratoconjunctivitis sicca (dry eye disease) associated with Meibomian gland dysfunction.

12. The method according to claim 1, wherein the patient is not responsive to treatment with artificial tears.

13. The method according to claim 1, wherein the patient is a female.

14. The method according to claim 1, wherein the ocular surface damage of the corneal region is determined by grading the central corneal region by fluorescein staining of the cornea.

15. The method according to claim 1, wherein the severity of dryness, the burning/stinging, the itching feeling, the sticky feeling, the blurred vision and/or the foreign body sensation are determined by the Eye Dryness Score on a visual analog scale (VAS) from 0% to 100% indicating the level of discomfort of the patient and wherein the reduction of frequency of dryness and/or the awareness of dryness is determined by the Eye Dryness Score on a visual analog scale (VAS) from 0% to 100% indicating the percentage of time said dryness symptoms are experienced by the patient, and wherein the total ocular surface disease index (OSDI) score is determined on a scale of 1 to 100 with higher scores representing greater disability of the patient.
